SEQUENCE LISTING (26 sequences)

SEQ ID NO: 1 - DNA, 35 bp, Artificial Sequence
  Description of Artificial Sequence Synthetic primer
cagctccatctacttccaaaccgtgctgagcgtgg

SEQ ID NO: 2 - DNA, 35 bp, Artificial Sequence
  Description of Artificial Sequence Synthetic primer
ccacgctcagcacggtttggaagtagatggagctg

SEQ ID NO: 3 - DNA, 33 bp, Artificial Sequence
  Description of Artificial Sequence Synthetic primer
ctgctgctcgggacagtggagagtggcaagagt

SEQ ID NO: 4 - DNA, 33 bp, Artificial Sequence
  Description of Artificial Sequence Synthetic primer
actcttgccactctccactgtcccgagcagcag

SEQ ID NO: 5 - DNA, 33 bp, Artificial Sequence
  Description of Artificial Sequence Synthetic primer
gatgtgcttagagttcaagtccccaccacaggg

SEQ ID NO: 6 - DNA, 33 bp, Artificial Sequence
  Description of Artificial Sequence Synthetic primer
ccctgtggtggggacttgaactctaagcacatc

SEQ ID NO: 7 - DNA, 33 bp, Artificial Sequence
  Description of Artificial Sequence Synthetic primer
gtcgatgtagggggcctaaggtcagagagaaga

SEQ ID NO: 8 - DNA, 33 bp, Artificial Sequence
  Description of Artificial Sequence Synthetic primer
tcttctctctgaccttaggccccctacatcgac

SEQ ID NO: 9 - DNA, 37 bp, Artificial Sequence
  Description of Artificial Sequence Synthetic primer
aacactcagcacggtctggaaataaatactgctgtac

SEQ ID NO: 10 - DNA, 37 bp, Artificial Sequence
  Description of Artificial Sequence Synthetic primer
gtacagcagtatttatttccagaccgtgctgagtgtt

SEQ ID NO: 11 - DNA, 29 bp, Artificial Sequence
  Description of Artificial Sequence Synthetic primer
tcttgccactctctactgtcccgagcagc

SEQ ID NO: 12 - DNA, 29 bp, Artificial Sequence
  Description of Artificial Sequence Synthetic primer
gctgctcgggacagtagagagtggcaaga

SEQ ID NO: 13 - DNA, 27 bp, Artificial Sequence
  Description of Artificial Sequence Synthetic primer
ccctgtggtggggacttgaactctaag

SEQ ID NO: 14 - DNA, 33 bp, Artificial Sequence; identical to SEQ ID NO: 5
  Description of Artificial Sequence Synthetic primer

SEQ ID NO: 15 - DNA, 31 bp, Artificial Sequence
  Description of Artificial Sequence Synthetic primer
cttctctctgaccttaggccccctacatcga

SEQ ID NO: 16 - DNA, 31 bp, Artificial Sequence
  Description of Artificial Sequence Synthetic primer
tcgatgtagggggcctaaggtcagagagaag

SEQ ID NO: 17 - DNA, 22 bp, Artificial Sequence
  Description of Artificial Sequence Synthetic primer
gagcacaataaggctcatgcac

SEQ ID NO: 18 - DNA, 20 bp, Artificial Sequence
  Description of Artificial Sequence Synthetic primer
ttgttgcgtaggcaggtagg

SEQ ID NO: 19 - DNA, 23 bp, Artificial Sequence
  Description of Artificial Sequence Synthetic primer
tatttcctgaccgtgctgagtgt

SEQ ID NO: 20 - DNA, 19 bp, Artificial Sequence
  Description of Artificial Sequence Synthetic primer
tgacatcagaagccgaaag

SEQ ID NO: 21 - DNA, 20 bp, Artificial Sequence
  Description of Artificial Sequence Synthetic primer
taggcaagagtgccttgacg

SEQ ID NO: 22 - DNA, 21 bp, Artificial Sequence
  Description of Artificial Sequence Synthetic primer
tgcttcctgtaggaatcctct

SEQ ID NO: 23 - DNA, 20 bp, Artificial Sequence
  Description of Artificial Sequence Synthetic primer
atttgggcaacgagaccgat

SEQ ID NO: 24 - DNA, 21 bp, Artificial Sequence
  Description of Artificial Sequence Synthetic primer
gttgatcctccatcaccacga

SEQ ID NO: 25 - DNA, 20 bp, Artificial Sequence
  Description of Artificial Sequence Synthetic primer
catgggcaagaagaagatcg

SEQ ID NO: 26 - DNA, 20 bp, Artificial Sequence
  Description of Artificial Sequence Synthetic primer
tccaaggggatatccacaga

(19) United States
(12) Patent Application Publication
(10) Pub. No.: US 2021/0403864 A1
(43) Pub. Date: Dec. 30, 2021

(54) METHODS AND COMPOSITIONS FOR TREATING ACTIVATED G-ALPHA Q MUTANT CANCERS OR MELANOCYTIC MALIGNANCIES

(71) Applicant: Memorial Sloan Kettering Cancer Center, New York, NY (US)

(72) Inventors: Yu Chen, New York, NY (US); Ping Chi, New York, NY (US); Tyler Hitchman, New York, NY (US)

(21) Appl. No.: 17/291,064

(22) PCT Filed: Nov. 5, 2019
(86) PCT No.: PCT/US19/59788

§ 371 Date: May 4, 2021

Related U.S. Application Data
(60) Provisional application No. 62/756,435, filed on Nov. 6, 2018.

(51) Int. Cl. C12N 5/071; C07K 14/47; C07K 14/705; C12Q 1/6886; A61K 35/36; A61P 35/00

(52) U.S. Cl. CPC C12N 5/0626; C07K 14/4702; C07K 14/705; C12Q 2600/156; A61K 35/36; A61P 35/00; C12Q 2600/158; C12Q 1/6886

(57) ABSTRACT

The present disclosure provides compositions and methods for assaying the effectiveness of a potential therapeutic agent for treatment of an activated GαQ mutant cancer (e.g., melanoma or angiosarcoma) or an activated GαQ mutant melanocytic malignancy (e.g., Portwine stain or Sturge-Weber syndrome). Also disclosed herein are methods for treating an activated GαQ mutant cancer (e.g., melanoma or angiosarcoma) or an activated GαQ mutant melanocytic malignancy (e.g., Portwine stain or Sturge-Weber syndrome) in a subject in need thereof.

### CROSS-REFERENCE TO RELATED APPLICATIONS

This application claims the benefit of and priority to U.S. Provisional Patent Application No. 62/756,435, filed Nov. 6, 2018, the entire contents of which are incorporated herein by reference.

### TECHNICAL FIELD

The present technology relates generally to compositions and methods for assaying the effectiveness of a potential therapeutic agent for treatment of an activated GαQ mutant cancer (e.g., melanoma or angiosarcoma) or an activated GαQ mutant melanocytic malignancy (e.g., Portwine stain or Sturge-Weber syndrome). Also disclosed herein are methods for treating an activated GαQ mutant cancer (e.g., melanoma or angiosarcoma) or an activated GαQ mutant melanocytic malignancy (e.g., Portwine stain or Sturge-Weber syndrome) in a subject in need thereof comprising administering to the subject an effective amount of cyclic depsipeptide YM-254890 or FR900359.

### BACKGROUND

The following description of the background of the present technology is provided simply as an aid in understanding the present technology and is not admitted to describe or constitute prior art to the present technology.

Uveal Melanoma (UVM) is the most common intraocular malignancy with approximately 3,000 new cases per year in the United States (Chattopadhyay C, et al., Cancer 2299-2312 (2016)). Metastatic UVM has a median survival of less than six months and a five year survival rate of 15% (Jovanovic P et al., Int J Clin Exp Pathol. 6(7):1230-1244 (2013)). UVM is characterized by aberrant activation of the heterotrimeric Gα-protein q (Gαq) pathway, most commonly in Gαq proteins, GNAQ and GNA11, and less frequently in the G protein-coupled receptor, CYSLTR2. Activating mutations in GNAQ, GNA1, and CYSLTR2 directly activate phospholipase C and downstream effectors including protein kinase C (PKC) and MAP kinase (Moore A R, et al. Cell Rep. 22(9):2455-2468 (2018); Chen X et al., Cancer Cell 31(5):685-696 (2017)). There are currently no effective systemic therapies for advanced uveal melanoma because of the difficulty of targeting aberrantly activated GTPases and their downstream signaling pathways. Therapeutic developments in UVM have focused on downstream pathways, including inhibitors of PKC and MEK, which have been unsuccessful to date Chattopadhyay C, et al., Cancer 2299-2312 (2016); Carvajal R D, et al., J Clin Oncol. 36(12):1232-1239 (2018); Luke J J, et al., Pigment Cell Melanoma Res. 28(2):135-47 (2015)). YM-254890 is an allosteric Gαq inhibitor that stabilizes the GDP-bound inactive configuration of wild-type Gαq. However, YM-254890 is believed to be ineffective against the most common Gαq^{Q209L/P} mutation, which is thought to be defective in GTPase activity (Takasaki J, et al., J Biol Chem. 279(12):47438-47445 (2004)).

Accordingly, there is an urgent need for methods that effectively treat activated Gαq mutant cancers or malignancies, such as advanced uveal melanoma.

### SUMMARY OF THE PRESENT TECHNOLOGY

In one aspect, the present disclosure provides a recombinant melanin producing cell comprising a non-endogenous expression vector comprising a Gag mutant gene that is operably linked to an expression control sequence, wherein the Gαq mutant gene is selected from the group consisting of GNAQ G48L, GNAQ G48V, GNAQ R183Q, GNAQ R183H, GNAQ R183G, GNA11 G48L, GNA11 G48V, GNA11 R183Q, GNA11 R183H, GNA11 R183G, and CYSLTR2 L129Q. The recombinant melanin producing cell may be a melanoblast or a melanocyte. Additionally or alternatively, in some embodiments, the recombinant melanin producing cell does not comprise a MAPK activating mutation. Examples of MAPK activating mutations include but are not limited to KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

Additionally or alternatively, in some embodiments, the expression vector is a plasmid, a cosmid, a bacmid, a bacterial artificial chromosome (BAC), a yeast artificial chromosome (YAC), or a viral vector. The expression control sequence operably linked to the Gαq mutant gene may be a native promoter of the Gαq mutant gene or a heterologous promoter. Additionally or alternatively, in some embodiments, the expression control sequence operably linked to the Gαq mutant gene is an inducible promoter or a constitutive promoter.

In one aspect, the present disclosure provides a method for identifying a candidate agent for treating an activated GαQ mutant cancer or an activated GαQ mutant melanocytic malignancy comprising (a) contacting recombinant melanin producing cells of the present technology with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting expression levels of IP1 and/or cyclin D1 in the recombinant melanin producing cells of step (a), wherein a reduction in IP1 and/or cyclin D1 expression levels in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In another aspect, the present disclosure provides a method for identifying a candidate agent for treating an activated GαQ mutant cancer or an activated GαQ mutant melanocytic malignancy comprising (a) contacting recombinant melanin producing cells of the present technology with a candidate agent, wherein the recombinant melanin producing cells comprise a first non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; (b) contacting recombinant non-melanin producing cells with the candidate agent, wherein the recombinant non-melanin producing cells comprise a second non-endogenous expression vector comprising the same Gαq mutation as the recombinant melanin producing cells of step (a); and (c) detecting IP1 expression levels in the recombinant melanin producing cells of step (a) and the recombinant non-melanin producing cells of step (b), wherein a reduction in IP1 expression levels in the recombinant melanin producing cells of step (a) compared to that observed in recombinant non-melanin producing cells of step (b) indicates that the candidate agent is effective in treating an activated GαQ mutant cancer or an activated GαQ mutant melanocytic malignancy. In certain embodiments, the method further comprises detecting phosphorylation of one or more of RASGRP3, CRAF, MEK, and ERK in the recombinant melanin producing cells of step (a) and the recombinant non-melanin producing cells of step (b). Additionally or alternatively, in some embodiments, the method further comprises detecting TPA-independent proliferation in the recombinant melanin producing cells of step (a) and the recombinant non-melanin producing cells of step (b).

In one aspect, the present disclosure provides a method for identifying a candidate agent for treating an activated GαQ mutant cancer or an activated GαQ mutant melanocytic malignancy comprising (a) contacting recombinant melanin producing cells with a candidate agent, wherein the recombinant melanin producing cells comprise a non-endogenous expression vector comprising a Gag mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting phosphorylation of one or more of RASGRP3, CRAF, MEK, and ERK in the recombinant melanin producing cells of step (a), wherein reduced phosphorylation in one or more of RASGRP3, CRAF, MEK, or ERK in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In another aspect, the present disclosure provides a method for identifying a candidate agent for treating an activated GαQ mutant cancer or an activated GαQ mutant melanocytic malignancy comprising (a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting TPA-independent proliferation of the recombinant melanin producing cells of step (a), wherein a reduction in TPA-independent proliferation in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In yet another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting expression levels of IP1 and/or cyclin D1 in the recombinant melanin producing cells of step (a), wherein a reduction in IP1 and/or cyclin D1 expression levels in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In one aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a first non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; (b) contacting recombinant non-melanin producing cells with the test amount of the candidate agent, wherein the recombinant non-melanin producing cells comprise a second non-endogenous expression vector comprising the same Gαq mutation as the recombinant melanin producing cells of step (a); and (c) detecting IP1 expression levels in the recombinant melanin producing cells of step (a) and the recombinant non-melanin producing cells of step (b), wherein a reduction in IP1 expression levels in the recombinant melanin producing cells of step (a) compared to that observed in recombinant non-melanin producing cells of step (b) indicates that the test amount of the candidate agent is effective in treating an activated GαQ mutant cancer or an activated GαQ mutant melanocytic malignancy. The method may further comprise detecting phosphorylation of one or more of RASGRP3, CRAF, MEK, and ERK in the recombinant melanin producing cells of step (a) and the recombinant non-melanin producing cells of step (b). Additionally or alternatively, in some embodiments, the method further comprises detecting TPA-independent proliferation in the recombinant melanin producing cells of step (a) and the recombinant non-melanin producing cells of step (b).

In another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNAT′ Q209R, and CYSLTR2 L129Q; and (b) detecting phosphorylation of one or more of RASGRP3, CRAF, MEK, and ERK in the recombinant melanin producing cells of step (a), wherein reduced phosphorylation in one or more of RASGRP3, CRAF, MEK, or ERK in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In yet another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNAT R183C, GNA11 Q209L, GNA11 Q209P, GNA11 Q209R, and CYSLTR2 L129Q; and (b) detecting TPA-independent proliferation of the recombinant melanin producing cells of step (a), wherein a reduction in TPA-independent proliferation in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In any of the above embodiments of the methods disclosed herein, the activated GαQ mutant cancer or the activated GαQ mutant melanocytic malignancy is a uveal melanoma, a cutaneous melanoma, a primary meningeal melanocytic tumor, an angiosarcoma, a Portwine stain, or Sturge-Weber syndrome. Additionally or alternatively, in any of the preceding embodiments of the methods disclosed herein, the activated GαQ mutant cancer or the activated GαQ mutant melanocytic malignancy does not harbor a MAPK activating mutation. Examples of MAPK activating mutations include but are not limited to KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61R}, NRAS^{Q61K}, NRAS^{Q61L}, BRAF^{V600K} or BRAF^{V600E}.

In one aspect, the present disclosure provides a method for treating an activated GαQ mutant cancer or an activated GαQ mutant melanocytic malignancy in a subject in need thereof comprising administering to the subject a therapeutically effective amount of YM-254890 or FR900359. In another aspect, the present disclosure provides a method for treating an activated GαQ mutant cancer or an activated GαQ mutant melanocytic malignancy in a subject in need thereof comprising administering to the subject a therapeutically effective amount of YM-254890 or FR900359 and a therapeutically effective amount of a MEK inhibitor. Examples of MEK inhibitors include, but are not limited to, trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. In certain embodiments of the methods disclosed herein, the MEK inhibitor and YM-254890 or FR900359 are administered sequentially, simultaneously, or separately. Additionally or alternatively, in some embodiments of the methods disclosed herein, the MEK inhibitor is administered orally, intranasally, intravenously, intramuscularly, intraperitoneally, subcutaneously, intrahepatically, intraarterially, intratumorally, rectally, intracranially, intrathecally, or topically. Additionally or alternatively, in some embodiments of the methods disclosed herein, YM-254890 or FR900359 is administered orally, intranasally, intravenously, intramuscularly, intraperitoneally, subcutaneously, intrahepatically, intraarterially, intratumorally, rectally, intracranially, intrathecally, or topically.

In any and all embodiments of the methods disclosed herein, the activated GαQ mutant cancer or activated GαQ mutant melanocytic malignancy may be a uveal melanoma, a cutaneous melanoma, a primary meningeal melanocytic tumor, an angiosarcoma, a Portwine stain, or Sturge-Weber syndrome. Additionally or alternatively, in some embodiments of the methods disclosed herein, the activated GαQ mutant cancer or activated GαQ mutant melanocytic malignancy comprises a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q. In any of the preceding embodiments of the methods disclosed herein, the activated GαQ mutant cancer or activated GαQ mutant melanocytic malignancy does not comprise a MAPK activating mutation such as KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61R}, NRAS^{Q61K}, NRAS^{Q61L}, BRAF^{V600K} or BRAF^{V600E} Additionally or alternatively, in some embodiments, the method further comprises sequentially, separately, or simultaneously administering one or more therapeutic agents selected from the group consisting of: alkylating agents, antimetabolites, natural products, hormones, platinum coordination complexes, anthracenedione, substituted urea, methyl hydrazine derivatives, and adrenocortical suppressants.

Also provided herein are kits for practicing the methods of the present technology.

### BRIEF DESCRIPTION OF THE DRAWINGS

FIG. 1(A) shows the oncoprint of Gag pathway mutations in 80 uveal melanoma (UVM) patients who have undergone MSK-IMPACT testing (Chen et al., J Mol Diagn. 17(3):251-64 (2015).

FIG. 1(B) shows the oncoprint of Gag pathway mutations in 188 UVM patients from five published cohorts including TCGA (Janes M R, et al., Cell 172(3):578-589 (2018)), Cancer Research UK (CRUK) (Zehir A et al., Nat Med. 23(6):703-713 (2017)), QIMR Berghofer Medical Research Institute (Robertson, A. et al., Cancer Cell 32(2):204-220 (2017)), University of Duisburg-Essen (UNI-UDE) (Furney S J et al., Cancer Discov. 3(10):1122-1129 (2013)) and MD Anderson Cancer Center/Massachusetts Eye and Ear Infirmary (MDACC/MEEI) (Johansson P, et al., Oncotarget 7(4):4624-4631 (2016)).

FIG. 1(C) shows the 3D structure of active GNAQ highlighting Gly48 of the P-loop, Arg183 of Switch 1, and Gln209 of Switch 2 (ball-and-stick). The magnesium ion is shown as a large sphere and water molecules as small spheres. GDP and AlF4⁻ are both shown as sticks.

FIG. 2(A) shows a workflow diagram of generating mutation dependent melan-a cells. Cells were transduced with a control vector (MSCV PURO), human cDNAs with WT or activating mutations found in UVM patients (CYSLTR2 and GNAQ), or downstream MAPK activating mutations (KRAS^{G12V} and BRAF^{V600E}) 12-O-tetradecanoyl-phorbol-13-acetate (TPA) was then withdrawn from the media to drive mutation dependence and cells were accessed for growth and phenotypic changes.

FIG. 2(B) shows the basal level of IP1 accumulation in melan-a cells. Vector and WT controls are cultured with TPA whereas the remaining samples were cultured in absence of TPA. Error bars, s.e.m. from triplicate samples. ***P<0.0005.

FIG. 2(C) shows a growth assay for melan-a cells in the absence of TPA for six days. Growth was assayed by Celltiter-glo 2.0 at day 1 (D1), day 3 (D3), and day 6 (D6). Fold increase in growth is shown relative to D1 cell number (top). Fold change in growth between D6 and D1 from growth assay is represented as a bar graph (bottom). Error bars, s.e.m. from triplicate samples. ***P<0.0005.

FIG. 2(D) shows bright field images (left) of cells after TPA withdrawal for 2 weeks and then spun down (right) to show pigmentation. Scale bar 100 μm.

FIG. 2(E) shows immunoblot analysis of melanocyte lineage markers (MITF, TRP2/DCT, and SOX10) upon TPA withdrawal for 1 week.

FIG. 3(A) shows RT-PCR validations of cDNA inserts transduced into melan-a cells by comparing expression of GNAQ. All samples were compared to expression of RPL27 and were done in triplicate. Error bars, s.d. from triplicate samples. **P<0.005.

FIG. 3(B) shows RT-PCR validations of cDNA inserts transduced into melan-a cells by comparing expression of CYSLTR2. All samples were compared to expression of RPL27 and were done in triplicate. Error bars, s.d. from triplicate samples. **P<0.005.

FIG. 3(C) shows RT-PCR validations of cDNA inserts transduced into melan-a cells by comparing expression of KRAS. All samples were compared to expression of RPL27 and were done in triplicate. Error bars, s.d. from triplicate samples. **P<0.005.

FIG. 3(D) shows RT-PCR validations of cDNA inserts transduced into melan-a cells by comparing expression of BRAF. All samples were compared to expression of RPL27 and were done in triplicate. Error bars, s.d. from triplicate samples. **P<0.005.

FIG. 4(A) shows IP1 accumulation assays in mutation dependent melan-a cells. Cells were treated with increasing concentrations of YM-254890 (YM) for 3 hours (top) and 24 hours (bottom). Error bars, s.e.m. from three biological replicates, each with three technical replicates.

FIG. 4(B) shows a Western blot analysis of downstream UVM signaling in melan-a cells treated with 500 nM YM at 0, 2, 4, 8, and 24 hours. The last sample for each cell line was also treated with TPA for 24 hours showing rescue of the pathway.

FIG. 4(C) shows the dose response of melan-a cells treated with YM for 5 days at increasing doses (readout by CellTiter glo 2.0). Data are expressed as the percentage relative light units (RLU) relative to that observed with vehicle. Error bars, s.e.m. from triplicate samples.

FIG. 5(A) shows IP1 accumulation assays done in HEK293T cells transfected with constructs at 11 ng/well. Cells were treated with YM at increasing doses for 24 hours. Error bars, s.e.m. from three biological replicates, each with three technical replicates.

FIG. 5(B) shows immunoblots of KRAS^{G12V} (left) and BRAF^{V600E} (right) expressing melan-a cells. Samples were treated with 500 nM YM for 0, 2, 4, 8, and 24 hours with the last lane being treatment plus TPA add back.

FIG. 5(C) shows the YM dose response of GNAQ^{Q209L} and CYSLTR2^{L129Q} driven cells in the presence of TPA assayed by Celltiter-glo 2.0 for 5 days (-TPA samples are from FIG. 4(C) for reference). Data are expressed as the percentage RLU relative to that observed with vehicle. Error bars, s.e.m. from triplicate samples.

FIG. 6(A) shows an IP′ accumulation assay with UVM cells and A375 cells (cutaneous BRAFV600E). Cells were treated with increasing concentrations of YM for 3 hours (top) and 24 hours (bottom). Error bars, s.e.m from three biological replicates, each with three technical replicates.

FIG. 6(B) shows Western blot analysis of downstream UVM signaling in UVM cells and A375 cells treated with 500 nM YM at 0, 2, 4, 8, and 24 hours.

FIG. 6(C) shows the dose response of UVM and A375 cells treated with YM for 5 days at increasing doses (readout by CellTiter glo 2.0). Data are expressed as the percentage RLU relative to that observed with vehicle. Error bars, s.e.m. from triplicate samples.

FIG. 7 shows YM dose response in non-Gq mutant UVM cell lines Mel285 and Mel290, cutaneous melanomas A375 (BRAF^{V600E} from FIG. 6(C) for reference) and SK-MEL-2 (NRAS^{Q61R}), and lung adenocarcinoma cells A549 (KRAS^{G12S}) treated for 5 days and assayed by Celltiter-glo 2.0. Data are expressed as the percentage RLU relative to that observed with vehicle. Error bars, s.e.m. from triplicate samples.

FIG. 8 shows the colony formation by BRAF-mutant A375 cutaneous melanoma cells (YM-254890 resistant and trametinib sensitive), and Gq-mutant Mel202 and uveal melanoma cells treated with YM-254890, trametinib, or both.

FIG. 9 shows the in vivo anti-tumor activity of (1) MEK162 (10 mg/kg oral twice daily), (2) YM-254890 (2.5 mg/kg intraperitoneally once daily) and (3) the combination of MEK162 (10 mg/kg oral twice daily) and YM-254890 (2.5 mg/kg intraperitoneally once daily) in comparison with vehicle only control in human Mel202 uveal melanoma xenografts in SCID mice. **** indicates p<0.0001.

FIG. 10 shows in vivo anti-tumor activity as measured by tumor volume change of GNAQ^{Q209L} transduced mouse melan-A cells grafted into C57/B6 mice 20 days after treatment. After the tumors were established, the mice were treated with (1) MEK162 (10 mg/kg oral twice daily), (2) YM-254890 (2.5 mg/kg intraperitoneally once daily), (3) the combination of MEK162 (10 mg/kg oral twice daily) and YM-254890 (2.5 mg/kg intraperitoneally once daily) or (4) vehicle only. Tumor volumes were measured 20 days after treatment. ** indicates p<0.01; ns=not significant.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Advances in targeted therapy have shown that direct inhibition of mutated oncogenes, such as EGFR, KIT and BRAF can be highly efficacious, whereas inhibition of downstream signaling is more challenging. UVM is molecularly defined by mutational activation of Gαq and harbors a very low mutational burden suggesting that effective Gαq inhibition has high therapeutic potential. Compared to kinases, rational design of drugs against GTPases, such as Gαq and RAS, has been challenging for a number of reasons: (a) activating mutations in GTPases are enzymatically defective whereas those in kinases are enzymatically hyperactive; and (b) GTPases activate downstream effectors through protein-protein interactions whereas kinases activate effectors through enzymatic modification. Therefore, GTPases cannot be targeted though inhibition of enzymatic activity but requires allosteric drugs that affect conformation or effector binding.

The present disclosure provides recombinant melanin producing cells comprising a non-endogenous expression vector that includes specific oncogenic driver mutations in GNAQ, GNA11 or CYSLTR2. The recombinant melanin producing cell compositions of the present technology are useful in methods for identifying candidate agents for treating an activated GαQ mutant cancer or an activated GαQ mutant melanocytic malignancy. For example, while YM-254890 has been previously reported to be ineffective against the most common Gαq^{Q2091-11}′ mutation, the recombinant GNAQ^{Q209} melanin producing cells disclosed herein exhibited sensitivity to YM-254890 treatment. These results demonstrate that these activating Gαq mutations cycle back to the GDP bound state and are amenable to allosteric inhibition.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples can also be obtained from biopsies of internal organs or from cancers. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, the term “gene” means a segment of DNA that contains all the information for the regulated biosynthesis of an RNA product, including promoters, exons, introns, and other untranslated regions that control expression.

As used herein, a “heterologous nucleic acid sequence” is any sequence placed at a location where it does not normally occur. A heterologous nucleic acid sequence may comprise a sequence that does not naturally occur in a cell, or it may comprise only sequences naturally found in the cell, but placed at a non-normally occurring location in the cell. In some embodiments, the heterologous nucleic acid sequence is not an endogenous sequence. In certain embodiments, the heterologous nucleic acid sequence is an endogenous sequence that is derived from a different cell. In other embodiments, the heterologous nucleic acid sequence is a sequence that occurs naturally in a cell but is then relocated to another site where it does not naturally occur, rendering it a heterologous sequence at that new site.

As used herein, the terms “individual”, “patient”, or “subject” can be an individual organism, a vertebrate, a mammal, or a human. In some embodiments, the individual, patient or subject is a human.

As used herein, “operably linked” means that expression control sequences are positioned relative to a nucleic acid of interest to initiate, regulate or otherwise control transcription of the nucleic acid of interest.

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the cell of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the cell of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a cell. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, a “synergistic” effect refers to a greater-than-additive therapeutic effect which is produced by a combination of at least two agents, and which exceeds that which would otherwise result from the individual administration of the at least two agents. For example, lower doses of one or more agents may be used in treating a disease or disorder, resulting in increased therapeutic efficacy and decreased side-effects.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, a “vector” refers to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome. Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

Recombinant Melanin Producing Cells of the Present Technology

In one aspect, the present disclosure provides a recombinant melanin producing cell comprising a non-endogenous expression vector comprising a Gαq mutant gene that is operably linked to an expression control sequence, wherein the Gαq mutant gene is selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q. The recombinant melanin producing cell may be a melanoblast or a melanocyte. Additionally or alternatively, in some embodiments, the recombinant melanin producing cell does not comprise a MAPK activating mutation. Examples of MAPK activating mutations include but are not limited to KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

The Gαq mutant gene sequence can be introduced as a linear construct, a circular plasmid, or a viral vector, which can be incorporated and inherited as a transgene integrated into the host genome, or as an extrachromosomal plasmid (Gassmann, et al., Proc. Natl. Acad. Sci. USA (1995) 92: 1292). The non-endogenous expression vector may be a DNA or RNA vector. Additionally or alternatively, in some embodiments, the expression vector is a plasmid, a cosmid, a bacmid, a bacterial artificial chromosome (BAC), a yeast artificial chromosome (YAC), or a viral vector.

Any viral vector capable of accepting the coding sequences for the transcript(s) to be expressed can be used, for example, vectors derived from adenovirus (AV); adeno-associated virus (AAV); retroviruses (e.g., lentiviruses (LV), Rhabdoviruses, murine leukemia virus); herpes virus, and the like. Viral vectors can be constructed based on, but not limited to, adeno-associated virus (for a review, see Muzyczka, et al, Curr. Topics Micro. Immunol. (1992) 158:97-129)); adenovirus (see, for example, Berkner, et al, BioTechniques (1998) 6:616), Rosenfeld et al. (1991, Science 252:431-434), and Rosenfeld et al. (1992), Cell 68: 143-155)); alphavirus as well as others known in the art. Retroviruses have been used to introduce a variety of genes into many different cell types, including epithelial cells, in vitro and/or in vivo (see, e.g., Eglitis, et al, Science (1985) 230: 1395-1398; Danos and Mulligan, Proc. Natl. Acad. Sci. USA (1998) 85:6460-6464; Wilson et al, 1988, Proc. Natl. Acad. Sci. USA 85:3014-3018; Armentano et al, 1990, Proc. Natl. Acad. Sci. USA 87:61416145; Huber et al, 1991, Proc. Natl. Acad. Sci. USA 88:8039-8043; Ferry et al, 1991, Proc. Natl. Acad. Sci. USA 88:8377-8381; Chowdhury et al, 1991, Science 254: 1802-1805; van Beusechem. et al, 1992, Proc. Natl. Acad. Sci. USA 89:7640-19; Kay et al, 1992, Human Gene Therapy 3:641-647; Dai et al, 1992, Proc. Natl. Acad. Sci. USA 89: 10892-10895; Hwu et al, 1993, 1 Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al, 1991, Human Gene Therapy 2:5-10; Cone et al, 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al, 1992, 1 Infectious Disease, 166:769).

The expression control sequence operably linked to the Gαq mutant gene may be a native promoter of the Gαq mutant gene or a heterologous promoter. Additionally or alternatively, in some embodiments, the expression control sequence operably linked to the Gαq mutant gene is an inducible promoter or a constitutive promoter. In some embodiments, the promoter driving transcription of the Gαq mutant gene within the expression vector may be a eukaryotic RNA polymerase I (e.g., ribosomal RNA promoter), RNA polymerase II (e.g., CMV early promoter or actin promoter or U1 snRNA promoter), RNA polymerase III promoter (e.g., U6 snRNA or 7SK RNA promoter), or a prokaryotic promoter (for example the T7 promoter, provided the expression plasmid also encodes T7 RNA polymerase required for transcription from a T7 promoter). In certain embodiments, the promoter directs tissue-specific or cell-specific expression. Additionally or alternatively, in some embodiments, transcription may be regulated by an inducible regulatory sequence such as a regulatory sequence that is sensitive to certain physiological regulators. Such inducible expression systems, suitable for the control of transgene expression in cells or in mammals include regulation by ecdysone, estrogen, progesterone, tetracycline, ampicillin, doxycycline, glucose, saccharides, chemical inducers of dimerization, isopropyl-beta-D-1-thiogalactopyranoside (IPTG) and the like. A person skilled in the art would be able to choose the appropriate regulatory/promoter sequence for the expression vector.

Successful introduction of expression vectors into host cells can be monitored using various known methods. Selection of expression vectors suitable for inserting nucleic acid sequences for expressing transcripts into the vector, and methods of delivering the vector to the cells of interest are within the skill in the art. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. The delivery of the expression vector containing recombinant DNA can by performed by abiologic or biologic systems including but not limited to liposomes, virus-like particles, transduction particles derived from phage or viruses, and conjugation.

Screening Methods Using Recombinant Melanin Producing Cells of the Present Technology

In one aspect, the present disclosure provides a method for identifying a candidate agent for treating an activated GαQ mutant cancer or an activated GαQ mutant melanocytic malignancy comprising (a) contacting recombinant melanin producing cells of the present technology with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNAT′ R183H, GNA11 R183G, GNA11 R183C, GNA11 Q209L, GNA11 Q209P, GNA11 Q209R, and CYSLTR2 L129Q; and (b) detecting expression levels of IP1 and/or cyclin D1 in the recombinant melanin producing cells of step (a), wherein a reduction in IP1 and/or cyclin D1 expression levels in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In another aspect, the present disclosure provides a method for identifying a candidate agent for treating an activated GαQ mutant cancer or an activated GαQ mutant melanocytic malignancy comprising (a) contacting recombinant melanin producing cells of the present technology with a candidate agent, wherein the recombinant melanin producing cells comprise a first non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; (b) contacting recombinant non-melanin producing cells with the candidate agent, wherein the recombinant non-melanin producing cells comprise a second non-endogenous expression vector comprising the same Gαq mutation as the recombinant melanin producing cells of step (a); and (c) detecting IP1 expression levels in the recombinant melanin producing cells of step (a) and the recombinant non-melanin producing cells of step (b), wherein a reduction in IP1 expression levels in the recombinant melanin producing cells of step (a) compared to that observed in recombinant non-melanin producing cells of step (b) indicates that the candidate agent is effective in treating an activated GαQ mutant cancer or an activated GαQ mutant melanocytic malignancy. In certain embodiments, the method further comprises detecting phosphorylation of one or more of RASGRP3, CRAF, MEK, and ERK in the recombinant melanin producing cells of step (a) and the recombinant non-melanin producing cells of step (b). Additionally or alternatively, in some embodiments, the method further comprises detecting TPA-independent proliferation in the recombinant melanin producing cells of step (a) and the recombinant non-melanin producing cells of step (b).

In one aspect, the present disclosure provides a method for identifying a candidate agent for treating an activated GαQ mutant cancer or an activated GαQ mutant melanocytic malignancy comprising (a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting phosphorylation of one or more of RASGRP3, CRAF, MEK, and ERK in the recombinant melanin producing cells of step (a), wherein reduced phosphorylation in one or more of RASGRP3, CRAF, MEK, or ERK in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In another aspect, the present disclosure provides a method for identifying a candidate agent for treating an activated GαQ mutant cancer or an activated GαQ mutant melanocytic malignancy comprising (a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and (b) detecting TPA-independent proliferation of the recombinant melanin producing cells of step (a), wherein a reduction in TPA-independent proliferation in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy.

In yet another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNAT′ Q209R, and CYSLTR2 L129Q; and (b) detecting expression levels of IP1 and/or cyclin D1 in the recombinant melanin producing cells of step (a), wherein a reduction in IP1 and/or cyclin D1 expression levels in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In one aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a first non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; (b) contacting recombinant non-melanin producing cells with the test amount of the candidate agent, wherein the recombinant non-melanin producing cells comprise a second non-endogenous expression vector comprising the same Gag mutation as the recombinant melanin producing cells of step (a); and (c) detecting IP1 expression levels in the recombinant melanin producing cells of step (a) and the recombinant non-melanin producing cells of step (b), wherein a reduction in IP1 expression levels in the recombinant melanin producing cells of step (a) compared to that observed in recombinant non-melanin producing cells of step (b) indicates that the test amount of the candidate agent is effective in treating an activated GαQ mutant cancer or an activated GαQ mutant melanocytic malignancy. The method may further comprise detecting phosphorylation of one or more of RASGRP3, CRAF, MEK, and ERK in the recombinant melanin producing cells of step (a) and the recombinant non-melanin producing cells of step (b). Additionally or alternatively, in some embodiments, the method further comprises detecting TPA-independent proliferation in the recombinant melanin producing cells of step (a) and the recombinant non-melanin producing cells of step (b).

In another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNAIJ R183Q, GNA11 R183H, GNA11 R183G, GNA11 R183C, GNA11 Q209L, GNA11 Q209P, GNAT′ Q209R, and CYSLTR2 L129Q; and (b) detecting phosphorylation of one or more of RASGRP3, CRAF, MEK, and ERK in the recombinant melanin producing cells of step (a), wherein reduced phosphorylation in one or more of RASGRP3, CRAF, MEK, or ERK in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In yet another aspect, the present disclosure provides a method for determining an effective amount of a candidate agent for treating an activated GαQ mutant cancer or an activated GαQ mutant melanocytic malignancy comprising (a) contacting recombinant melanin producing cells with a test amount of a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNAT R183C, GNA11 Q209L, GNA11 Q209P, GNA11 Q209R, and CYSLTR2 L129Q; and (b) detecting TPA-independent proliferation of the recombinant melanin producing cells of step (a), wherein a reduction in TPA-independent proliferation in the recombinant melanin producing cells of step (a) compared to that observed in recombinant melanin producing cells that are not contacted with the candidate agent indicates that the test amount of the candidate agent is effective in treating an activated GαQ mutant cancer or an activated GαQ mutant melanocytic malignancy.

In any of the above embodiments of the methods disclosed herein, the activated GαQ mutant cancer or activated GαQ mutant melanocytic malignancy is a uveal melanoma, a cutaneous melanoma, a primary meningeal melanocytic tumor, an angiosarcoma, a Portwine stain, or Sturge-Weber syndrome. Additionally or alternatively, in any of the preceding embodiments of the methods disclosed herein, the activated GαQ mutant cancer or activated GαQ mutant melanocytic malignancy does not harbor a MAPK activating mutation. Examples of MAPK activating mutations include but are not limited to KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

Methods for Treating Activated GαO Mutant Cancers or Activated GαO Mutant Melanocytic Malignancy

YM-254890 (YM) is a naturally-occurring cyclic depsipeptide first studied for its ability to inhibit platelet aggregation by perturbing Gαq-mediated Ca′ mobilization (Takasaki J, et al., J Biol Chem. 279(12):47438-47445 (2004)). YM is an allosteric inhibitor that binds to the hydrophobic cleft between two inter-domain linkers of wild-type Gαq, stabilizing the inactive GDP-bound form by hindering the flexibility of the linkers (Nishimura A, et al., Proc Natl Acad Sci. 107(31):13666-71 (2010)). However, initial studies using overexpression systems indicated that YM was unable to inhibit aberrant Gαq signaling downstream of GαqQ^{209L} (Takasaki J, et al., J Biol Chem. 279(12):47438-47445 (2004)), which is the most commonly occuring mutation in uveal melanoma. FR900359 (FR) is a distinct cyclic depsipeptide from the plant Ardisia crenata that inhibits Gq proteins.

The cyclic depsipeptide compositions (e.g., YM-254890, FR900359) of the present technology are useful for the treatment of an activated GαQ mutant cancer or an activated GαQ mutant melanocytic malignancy. Such treatment can be used in patients identified as exhibiting aberrant activation of the heterotrimeric Gα-protein q (Gαq) pathway. In one aspect, the present disclosure provides a method for treating an activated GαQ mutant cancer or an activated GαQ mutant melanocytic malignancy in a subject in need thereof comprising administering to the subject a therapeutically effective amount of a cyclic depsipeptide composition disclosed herein. In another aspect, the present disclosure provides a method for treating an activated GαQ mutant cancer or an activated GαQ mutant melanocytic malignancy in a subject in need thereof comprising administering to the subject a therapeutically effective amount of YM-254890 or FR900359 and a therapeutically effective amount of a MEK inhibitor. Examples of MEK inhibitors include, but are not limited to, trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327. In certain embodiments of the methods disclosed herein, the MEK inhibitor and YM-254890 or FR900359 are administered sequentially, simultaneously, or separately. Additionally or alternatively, in some embodiments of the methods disclosed herein, the MEK inhibitor is administered orally, intranasally, intravenously, intramuscularly, intraperitoneally, subcutaneously, intrahepatically, intraarterially, intratumorally, rectally, intracranially, intrathecally, or topically. Additionally or alternatively, in some embodiments of the methods disclosed herein, YM-254890 or FR900359 is administered orally, intranasally, intravenously, intramuscularly, intraperitoneally, subcutaneously, intrahepatically, intraarterially, intratumorally, rectally, intracranially, intrathecally, or topically.

In any and all embodiments of the methods disclosed herein, the activated GαQ mutant cancer or activated GαQ mutant melanocytic malignancy may be a uveal melanoma, a cutaneous melanoma, a primary meningeal melanocytic tumor, an angiosarcoma, a Portwine stain, or Sturge-Weber syndrome. Additionally or alternatively, in some embodiments of the methods disclosed herein, the activated GαQ mutant cancer or activated GαQ mutant melanocytic malignancy comprises a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q. In any of the preceding embodiments of the methods disclosed herein, the activated GαQ mutant cancer or activated GαQ mutant melanocytic malignancy does not comprise a MAPK activating mutation such as KRAS^{G12V}, KRAS^{G12D}, KRAS^{G13C}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

The cyclic depsipeptide compositions of the present technology, alone or in combination with MEK inhibitors, may be employed in conjunction with other therapeutic agents useful in the treatment of an activated GαQ mutant cancer or an activated GαQ mutant melanocytic malignancy. For example, the cyclic depsipeptide compositions of the present technology may be separately, sequentially or simultaneously administered with at least one additional therapeutic agent selected from the group consisting of alkylating agents, antimetabolites, natural products, and hormones. Other agents that can be used in the therapeutic methods described herein include platinum coordination complexes (e.g., cisplatin, carboblatin), anthracenedione (e.g., mitoxantrone), substituted urea (e.g., hydroxyurea), methyl hydrazine derivative (e.g., procarbazine), and adrenocortical suppressant (e.g., mitotane, aminoglutethimide). Examples of alkylating agents include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones include but are not limited to adrenocorticosteroids (e.g, prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). In some embodiments, the at least one additional therapeutic agent is a chemotherapeutic agent. Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), bevacizumab, oxaliplatin, melphalan, etoposide, mechlorethamine, bleomycin, microtubule poisons, annonaceous acetogenins, or combinations thereof.

The compositions of the present technology may optionally be administered as a single bolus to a subject in need thereof. Alternatively, the dosing regimen may comprise multiple administrations performed at various times after the appearance of tumors.

Administration can be carried out by any suitable route, including orally, intranasally, parenterally (intravenously, intramuscularly, intraperitoneally, or subcutaneously), intrahepatically, intraarterially, intratumorally, rectally, intracranially, intrathecally, or topically. Administration includes self-administration and the administration by another. It is also to be appreciated that the various modes of treatment of medical conditions as described are intended to mean “substantial”, which includes total but also less than total treatment, and wherein some biologically or medically relevant result is achieved.

In some embodiments, the cyclic depsipeptides of the present technology comprise pharmaceutical formulations which may be administered to subjects in need thereof in one or more doses. Dosage regimens can be adjusted to provide the desired response (e.g., a therapeutic response).

Typically, an effective amount of the cyclic depsipeptide compositions of the present technology, sufficient for achieving a therapeutic effect, range from about 0.000001 mg per kilogram body weight per day to about 10,000 mg per kilogram body weight per day. Typically, the dosage ranges are from about 0.0001 mg per kilogram body weight per day to about 100 mg per kilogram body weight per day. For administration of cyclic depsipeptides, the dosage ranges from about 0.0001 to 100 mg/kg, and more usually 0.01 to 5 mg/kg every week, every two weeks or every three weeks, of the subject body weight. For example, dosages can be 1 mg/kg body weight or 10 mg/kg body weight every week, every two weeks or every three weeks or within the range of 1-10 mg/kg every week, every two weeks or every three weeks.

In one embodiment, a single dosage of cyclic depsipeptide ranges from 0.1-10,000 micrograms per kg body weight. In one embodiment, cyclic depsipeptide concentrations in a carrier range from 0.2 to 2000 micrograms per delivered milliliter. An exemplary treatment regime entails administration once per every two weeks or once a month or once every 3 to 6 months. Cyclic depsipeptides may be administered on multiple occasions. Intervals between single dosages can be hourly, daily, weekly, monthly or yearly. Intervals can also be irregular as indicated by measuring blood levels of the cyclic depsipeptide in the subject. In some methods, dosage is adjusted to achieve a serum cyclic depsipeptide concentration in the subject of from about 75 μg/mL to about 125 μg/mL, 100 μg/mL to about 150 μg/mL, from about 125 μg/mL to about 175 vg/mL, or from about 150 μg/mL to about 200 μg/mL. Alternatively, cyclic depsipeptides can be administered as a sustained release formulation, in which case less frequent administration is required. Dosage and frequency vary depending on the half-life of the cyclic depsipeptide in the subject. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

Formulations of Pharmaceutical Compositions

According to the methods of the present technology, the cyclic depsipeptides disclosed herein can be incorporated into pharmaceutical compositions suitable for administration. The pharmaceutical compositions generally comprise the cyclic depsipeptides disclosed herein and a pharmaceutically-acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A pharmaceutical composition of the present technology is formulated to be compatible with its intended route of administration. The cyclic depsipeptide compositions of the present technology can be administered by parenteral, topical, intravenous, oral, subcutaneous, intraarterial, intradermal, transdermal, rectal, intrathecal, intraperitoneal, intranasal; or intramuscular routes, or as inhalants. The cyclic depsipeptides can optionally be administered in combination with other agents that are at least partly effective in treating an activated GαQ mutant cancer or an activated GαQ mutant melanocytic malignancy.

The cyclic depsipeptides may be administered directly or in the form of suitable preparations, enterally, parenterally, dermally, nasally, by environment treatment, or with the aid of active-compound-containing shaped articles such as, for example, strips, plates, bands, collars, ear marks, limb bands, or marking devices. The cyclic depsipeptides may be administered enterally, for example orally, in the form of powders, tablets, capsules, passes, drinks, granules, or solutions, suspensions and emulsions which can be administered orally, or via boli, medicated feed or drinking water. Dermal administration is effected, for example, in the form of dipping, spraying or pouring-on and spotting-on. Parenteral administration is effected, for example, in the form of an injection (intramuscular, subcutaneous, intravenous, intraperitoneal) or by implants.

Suitable preparations include solutions such as injectable solutions, oral solutions, concentrates for oral administration after dilution, solutions for use on the skin or in body cavities, pour-on and spot-on formulations, gels; emulsions and suspensions for oral or dermal administration and for injection; semi-solid preparations; formulations in which the active compound (e.g., the cyclic depsipeptides disclosed herein) is incorporated in a cream base or in an oil-in-water or water-in-oil emulsion base; solid preparations such as powders, premixes or concentrates, granules, pellets, tablets, boli, capsules; aerosols and inhalants, shaped articles containing cyclic depsipeptides.

Injectable solutions may be administered intravenously, intramuscularly or subcutaneously. Injectable solutions can be prepared by dissolving the active compound (e.g., the cyclic depsipeptides disclosed herein) in a suitable solvent and, if appropriate, adding additives such as solubilizers, acids, bases, buffer salts, antioxidants and preservatives. The solutions may be sterile-filtered and packaged. Examples of physiologically acceptable solvents include but are not limited to, water, alcohols such as ethanol, butanol, benzyl alcohol, glycerol, propylene glycol, polyethylene glycols, N-methylpyrrolidone, or any mixture thereof. If appropriate, the active compounds can also be dissolved in physiologically acceptable vegetable or synthetic oils which are suitable for injection. Examples of solubilizers (solvents which enhance solution of the active compound in the main solvent, or which prevent its precipitation) include but are not limited to, polyvinylpyrrolidone, polyoxyethylated castor oil, and polyoxyethylated sorbitan esters. Examples of preservatives include but are not limited to, benzyl alcohol, trichlorobutanol, p-hydroxybenzoic esters, and n-butanol.

Oral compositions may be prepared as described above in the case of the injectable solutions. Oral compositions generally include an inert diluent or an edible carrier. They can be enclosed in gelatin capsules or compressed into tablets. For the purpose of oral therapeutic administration, the active compound (e.g., the cyclic depsipeptides disclosed herein) can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

Solutions for use on the skin may be applied dropwise, brushed on, rubbed in, splashed on or sprayed on. These solutions are prepared as described above in the case of injectable solutions. It may be advantageous to add thickeners during the preparation. Thickeners include but are not limited to, inorganic thickeners such as bentonites, colloidal silica, aluminium monostearate, organic thickeners such as cellulose derivatives, polyvinyl alcohols and their copolymers, acrylates and methacrylates. Gels may be applied to, or brushed onto, the skin, or introduced into body cavities. Gels can be prepared by treating solutions which have been prepared as described in the case of the injectable solutions with such an amount of thickener that a clear substance of cream-like consistency is formed. Thickeners employed include the thickeners described herein.

Pour-on and spot-on formulations are poured onto, or splashed onto, limited areas of the skin, the active compound (e.g., the cyclic depsipeptides disclosed herein) penetrating the skin and acting systemically. Pour-on and spot-on formulations are prepared by dissolving, suspending or emulsifying the active compound in suitable solvents or solvent mixtures which are tolerated by the skin. If appropriate, other adjuvants such as colourants, absorption accelerators, antioxidants, light stabilizers and tackifiers may be added. Examples of solvents include but are not limited to: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol monobutyl ether, ketones such as acetone, methyl ethyl ketone, aromatic and/or aliphatic hydrocarbons, vegetable or synthetic oils, DMF, dimethylacetamide, N-methylpyrrolidone, 2,2-dimethyl-4-oxy-methylene-1,3-dioxolane.

Examples of absorption accelerators include DMSO, spreading oils such as isopropyl myristate, dipropylene glycol pelargonate, silicone oils, fatty acid esters, triglycerides, and fatty alcohols such as isotridecyl alcohol, 2-octyldodecanol, cetylstearyl alcohol, and oleyl alcohol. Examples of fatty acid esters include ethyl stearate, di-n-butyryl adipate, hexyl laurate, dipropylene glycol pelargonate, esters of a branched fatty acid of medium chain length with saturated fatty alcohols of chain length C16-C18, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck preen fat, dibutyl phthalate, diisopropyl adipate, and ester mixtures related to the latter, etc.

Examples of antioxidants include sulphites or metabisulphites such as potassium metabisulphite, ascorbic acid, butylhydroxy-toluene, butylhydroxyanisole, and tocopherol. Examples of light stabilizers include novantisolic acid. Examples of tackifiers include cellulose derivatives, starch derivatives, polyacrylates, natural polymers such as alginates, gelatine.

Emulsions can be administered orally, dermally or in the form of injections, and are either of the water-in-oil type or of the oil-in-water type. Emulsions are prepared by dissolving the active compound (e.g., the cyclic depsipeptides disclosed herein) either in the hydrophobic or in the hydrophilic phase and homogenising this phase with the solvent of the other phase, with the aid of suitable emulsifiers and, if appropriate, other adjuvants such as colourants, absorption accelerators, preservatives, antioxidants, light stabilizers, and viscosity-increasing substances.

Examples of hydrophobic phase (oils) include: paraffin oils, silicone oils, natural vegetable oils such as sesame seed oil, almond oil, castor oil, oleic acid, synthetic triglycerides such as caprylic/capric acid biglyceride, triglyceride mixture with vegetable fatty acids of chain length C8-12 or with other specifically selected natural fatty acids, partial glyceride mixtures of saturated or unsaturated fatty acids which may also contain hydroxyl groups, and mono- and diglycerides of the C₈/C₁₀-fatty acids. Examples of hydrophilic phase include: water, alcohols such as, for example, propylene glycol, glycerol, sorbitol and their mixtures. Examples of emulsifiers include: non-ionic surfactants, for example polyoxyethylated castor oil, polyoxyethylated sorbitan monooleate, sorbitan monostearate, glycerol monostearate, polyoxyethyl stearate, alkylphenol polyglycol ethers; ampholytic surfactants such as disodium N-lauryl-13-iminodipropionate or lecithin; and anionic surfactants such as sodium lauryl sulphate, fatty alcohol ether sulphates, the monoethynolamine salt of mono/dialkyl polyglycol ether orthophosphoric esters.

Examples of other adjuvants include: viscosity-increasing substances and substances which stabilize the emulsion, such as carboxymethylcellulose, methylcellulose and other cellulose and starch derivatives, polyacrylates, alginates, gelatine, gum arabic, polyvinylpyrrolidone, polyvinyl alcohol, copolymers of methyl vinyl ether and maleic anhydride, polyethylene glycols, waxes, colloidal silica, or mixtures of the substances mentioned.

Suspensions can be administered orally, dermally or in the form of an injection. Suspensions can be prepared by suspending the active compound (e.g., the cyclic depsipeptides disclosed herein) in an excipient liquid, if appropriate with the addition of further adjuvants such as wetting agents, colourants, absorption accelerators, preservatives, antioxidants light stabilizers. Excipient liquids may be any homogeneous solvent and/or solvent mixture described herein. Wetting agents (dispersants) may be any surfactant described herein.

Semi-solid preparations can be administered orally or dermally and are distinguishable from the above-described suspensions and emulsions by their higher viscosity. To prepare solid preparations, the active compound (e.g., the cyclic depsipeptides disclosed herein) is mixed with suitable excipients, if appropriate with the addition of adjuvants (e.g., preservatives, antioxidants, colourants), and the mixture is formulated as desired. Examples of suitable excipients include sodium chloride, carbonates such as calcium carbonate, hydrogen carbonates, aluminium oxides, silicas, clays, precipitated or colloidal silicon dioxide, phosphates, sugars, cellulose, foods and animal feeds such as dried milk, carcass meals, cereal meals and coarse cereal meals, and starches. Other suitable adjuvants are lubricants and glidants such as, for example, magnesium stearate, stearic acid, talc, bentonites, disintegrants such as starch or crosslinked polyvinylpyrrolidone, binders such as, for example, starch, gelatine or linear polyvinylpyrrolidone, and also dry binders such as microcrystalline cellulose.

For administration by inhalation, the cyclic depsipeptides disclosed herein are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the cyclic depsipeptides disclosed herein is formulated into ointments, salves, gels, or creams as generally known in the art.

The cyclic depsipeptides disclosed herein can also be prepared as pharmaceutical compositions in the form of suppositories (e.g., with conventional suppository bases such as cocoa butter and other glycerides) or retention enemas for rectal delivery.

In one embodiment, the cyclic depsipeptides disclosed herein are prepared with carriers that will protect the cyclic depsipeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically-acceptable carriers. These can be prepared according to methods known to those skilled in the art, e.g., as described in U.S. Pat. No. 4,522,811.

Kits

The present technology provides kits for use in any of the methods described herein. In one aspect, the present disclosure provides kits including any of the recombinant melanin producing cells disclosed herein and instructions for assaying the effectiveness of a candidate agent for treatment of an activated GαQ mutant cancer or an activated GαQ mutant melanocytic malignancy. In another aspect, the kits may include non-endogenous expression vectors comprising any Gαq mutation disclosed herein, mammalian host cells, and instructions for transforming the non-endogenous expression vectors into the mammalian host cells and using the transformed cells to identify a candidate agent for treating an activated GαQ mutant cancer or an activated GαQ mutant melanocytic malignancy. The mammalian host cells may be recombinant, non-recombinant, melanin producing, or non-melanin producing. In any of the embodiments disclosed herein, the kits can also comprise, e.g., a buffering agent, a preservative, a stabilizing agent, cell culture medium, cell culture supplements and the like. The kits of the present technology can further comprise components necessary for detecting expression levels and/or activity of one or more GαQ pathway biomarkers such as IP1, cyclin D1, p-RASGRP3, p-CRAF, p-MEK, and p-ERK. The kits can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit, e.g., for screening for a candidate agent that effectively treats an activated GαQ mutant cancer or an activated GαQ mutant melanocytic malignancy. In certain embodiments, the use of the reagents can be according to the methods of the present technology.

In another aspect, the present disclosure provides kits comprising a cyclic depsipeptide disclosed herein (e.g., YM-254890 or FR900359) and instructions for use to treat an activated GαQ mutant cancer or an activated GαQ mutant melanocytic malignancy. The kit may further comprise a description of selection of an individual suitable for treatment. Instructions supplied in the kits of the present disclosure may be written instructions on a label or package insert (e.g., a paper sheet included in the kit), or machine-readable instructions (e.g., instructions carried on a magnetic or optical storage disk). The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, etc. The kits may optionally include instructions customarily included in commercial packages of therapeutic products, that contain information about, for example, the indications, usage, dosage, manufacture, administration, contraindications and/or warnings concerning the use of such therapeutic products.

In any and all embodiments of the kits disclosed herein, the activated GαQ mutant cancer or the activated GαQ mutant melanocytic malignancy is a uveal melanoma, a cutaneous melanoma, a primary meningeal melanocytic tumor. an angiosarcoma, a Portwine stain, or Sturge-Weber syndrome.

### EXAMPLES

The present technology is further illustrated by the following Examples, which should not be construed as limiting in any way.

### Example 1: Materials and Methods

Reagents. YM-254890 was purchased from Wako Pure Chemical Industries (Richmond, Va., CAT#257-00631).

Exogenous Gene Expression. HEK293T cells were transiently transfected to express vectors containing synthetic CYSLTR2 (Moore A R, et al., Nat Genet. 48(6):675 (2016)) and GNAQ. Genewiz was used to codon optimize these sequences for expression in mammalian cells. The human GNAQ α-subunit plasmids encoding for GNAQ mutants Q209L, R183Q and G48V were generated by Quick Change (Agilent Technologies, Santa Clara, Calif.) site-directed mutagenesis. HEK293T cells were transiently transfected with the plasmids encoding for GNAQ WT and mutants subcloned into pcDNA3.1(+) using Lipofectamine according to the manufacturer's instructions. Briefly, 9,000 HEK293T cells were transfected in low-volume 384-well plates with 11 ng of total DNA/well for 24 hours. Human cDNAs for WT CYSLTR2 and GNAQ were obtained from Origene and cloned into MSCV-Puro (Addgene plasmid #68469) for stable expression in melan-a cells via retroviral transduction with X-tremeGENE 9 (Sigma-Aldrich, St. Louis Mo.). Quikchange primers were used to introduce the various mutant constructs. All constructs were confirmed by sequencing. The primers used in the experiments described herein are provided below:

| | Site-Directed | | |
|---|---|---|---|
| Gene | Mutation | Forward Primer | Reverse Primer |
| Synthetic | c.386T > A | CAGCTCCATCTACTTCCAAACC | CCACGCTCAGCACGGTTTGGAA |
| CYSLTR2 | | GTGCTGAGCGTGG | GTAGATGGAGCTG |
| L129Q | | (SEQ ID NO: 1) | (SEQ ID NO: 2) |
| Quickchange | | | |
| Synthetic | c.143G > T | CTGCTGCTCGGGACAGTGGAGA | ACTCTTGCCACTCTCCACTGTC |
| GNAQ | | GTGGCAAGAGT | CCGAGCAGCAG |
| G48V | | (SEQ ID NO: 3) | (SEQ ID NO: 4) |
| Quickchange | | | |
| Synthetic | c.548G > A | GATGTGCTTAGAGTTCAAGTCC | CCCTGTGGTGGGGACTTGAACT |
| GNAQ | | CCACCACAGGG | CTAAGCACATC |
| R183Q | | (SEQ ID NO: 5) | (SEQ ID NO: 6) |
| Quickchange | | | |
| Synthetic | c.626A > T | GTCGATGTAGGGGGCCTAAGGT | TCTTCTCTCTGACCTTAGGCCC |
| GNAQ | | CAGAGAGAAGA | CCTACATCGAC |
| Q290L | | (SEQ ID NO: 7) | (SEQ ID NO: 8) |
| Quickchange | | | |
| CYSLTR2 | c.386T > A | AACACTCAGCACGGTCTGGAAA | GTACAGCAGTATTTATTTCCAG |
| L192Q | | TAAATACTGCTGTAC | ACCGTGCTGAGTGTT |
| Quickchange | | (SEQ ID NO: 9) | (SEQ ID NO: 10) |
| GNAQ | c.143G > T | TCTTGCCACTCTCTACTGTCCC | GCTGCTCGGGACAGTAGAGAGT |
| G48V | | GAGCAGC | GGCAAGA |
| Quickchange | | (SEQ ID NO: 11) | (SEQ ID NO: 12) |
| GNAQ | c.548G > A | CCCTGTGGTGGGGACTTGAACT | GATGTGCTTAGAGTTCAAGTCC |
| R183Q | | CTAAG | CCACCACAGGG |
| Quickchange | | (SEQ ID NO: 13) | (SEQ ID NO: 14) |
| GNAQ | c.626A > T | CTTCTCTCTGACCTTAGGCCCC | TCGATGTAGGGGGCCTAAGGTC |
| Q209L | | CTACATCGA | AGAGAGAAG |
| Quickchange | | (SEQ ID NO: 15) | (SEQ ID NO: 16) |

Cell Culture and in vitro analysis. Mutation dependent melan-a cells were generated by transducing cells with cDNAs in MSCV-PURO (retrovirus) and then selecting cells with puromycin (1 μg/ml) for two days. TPA (Sigma-Aldrich, St. Louis, Mo.) was then withdrawn from cultures. Cells with activating mutations (CYSLTR2^{L129Q}, GNAQ^{G48V/R183Q/Q209L}, KRAS^{G12V}, and BRAF^{V600E}) continued to proliferate over several passages (>2 weeks) and were then validated with qRT-PCR and immunoblots. Vector and WT controls were unable to maintain proliferation after a couple passages upon TPA withdrawal. All cells were cultured in media containing 10% FBS, L-glutamine (2 nM), penicillin (100 U/ml), streptomycin (100 μg/ml). Melan-a cells provided by D. Bennett, St. George's Hospital, University of London (Johnson C P, et al., PLoS One. 12(6):1-17 (2017)), were cultured in RPMI supplemented with 200 nM TPA unless otherwise noted. HEK293T (from ATCC, Manassas, Va.) cells were cultured in DMEM-Q and UVM cells were cultured in RPMI. All cells tested negative for mycoplasma. Cell growth assays and YM dose response curves were assayed using CellTiter-glo 2.0 (Promega, Madison, Wis.) at given time points or after five days of incubation with YM and readout on a Glomax Luminometer (Promega, Madison, Wis.). IC₅₀ values were calculated using Prism 7.0 software. Growth assays were compared to day 1 readings and dose response curves were compared to vehicle. Data shown is representative of at least three independent experiments with at least three technical replicates. Dose-response curves and growth assays were analyzed using GraphPad Prism 7.0 software.

Immunoblotting. Lysates were harvested using SDS lysis buffer (50 mM Tris-HCL, pH 6.8, 1 mM EDTA, 150 mM NaCl, 1% SDS, 1 nM NaF) supplemented with fresh proteinase and phosphatase inhibitors (PhosSTOP, Roche, Basel, Switzerland; Complete EDTA-Free, Roche, Basel, Switzerland) and 1% PMSF (Sigma-Aldrich, St. Louis, Mo.). Lysates were incubated on ice for 30 minutes, sonicated for 15 minutes, and then centrifuged at max speed for 10 minutes at 4° C. Protein levels were quantified using BCA protein assay (Thermo Scientific, Waltham, Mass.). NuPAGE LDS Sample Buffer (4×) was added to a final concentration of 1×. Samples were run on NuPAGE Novex 4-12% Bis-Tris protein gels (Life Technologies, Carlsbad, Calif.), transferred electrophoretically to 0.45 μm PVDF membranes (Immobilon, EMD Millipore, Burlington, Mass.), and then blocked with StartingBlock blocking buffer (Thermo Scientific, Waltham, Mass.) for 1 hour at room temperature. Primary antibodies were incubated at 4° C. overnight in StartingBlock buffer at 1:1,000 dilution, unless noted otherwise. Primary Antibodies used in the experiments described herein are provided below:

| Target Protein | Catalog # | Company |
|---|---|---|
| GAPDH (1:5, 000) | 60004-I-Ig | Proteintech (Rosemont, IL) |
| MITF | 12590S | CST (Danvers, MA) |
| RASGRP3 | 3334S | CST (Danvers, MA) |
| CRAF | 53745S | CST (Danvers, MA) |
| p-CRAF (S338) | 9427S | CST (Danvers, MA) |
| MEK1/2 | 4694S | CST (Danvers, MA) |
| p-MEK1/2 (S217/221) | 9154S | CST (Danvers, MA) |
| ERK1/2 | 4696S | CST (Danvers, MA) |
| p-ERK1/2 (T202/Y204) | 4370S | CST (Danvers, MA) |
| Cyclin D1 | 2922S | CST (Danvers, MA) |
| p-RASGRP3 (T133) | ab124823 | Abcam (Cambridge, UK) |
| GP100 | ab137078 | Abcam (Cambridge, UK) |
| TRP2/DCT | ab74073 | Abcam (Cambridge, UK) |
| SOX10 | ab212843 | Abcam (Cambridge, UK) |

RT-PCR. RNA was extracted from cells using the E.Z.N.A Total RNA kit (Omega Bio-tek, Norcross, Ga.). 2 μg of RNA from each sample was reverse transcribed to cDNA using the High Capacity cDNA Reverse Transcription Kit (Applied Biosystems, Foster City, Calif.). Power SYBR Green PCR Master Mix (Applied Biosystems, Foster City, Calif.) was then used for PCR on a QuantStudio 6 Flex System (Applied Biosystems, Foster City, Calif.). Expression was normalized to ribosomal protein RPL27. Relative expression of mRNA was plotted as 2-ΔΔCt and each experiment was performed in triplicate and repeated in at least three independent experiments. Primers used in RT-PCR experiments are provided below:

| Primer | | |
|---|---|---|
| Target | Forward Primer | Reverse Primer |
| hGNAQ | GAGCACAATAAGGCT | TTGTTGCGTAGGCAGGTAGG |
| | CATGCAC | (SEQ ID NO: 18) |
| | (SEQ ID NO: 17) | |
| hCYSLTR2 | TATTTCCTGACCGTG | TGACATCAGAAGCCGAAAG |
| | CTGAGTGT | (SEQ ID NO: 20) |
| | (SEQ ID NO: 19) | |
| hKRAS | TAGGCAAGAGTGCCT | TGCTTCCTGTAGGAATCCTCT |
| | TGACG | (SEQ ID NO: 22) |
| | (SEQ ID NO: 21) | |
| hBRAF | ATTTGGGCAACGAGA | GTTGATCCTCCATCACCACGA |
| | CCGAT | (SEQ ID NO: 24) |
| | (SEQ ID NO: 23) | |
| hRPL27 | CATGGGCAAGAAGAA | TCCAAGGGGATATCCACAGA |
| | GATCG | (SEQ ID NO: 26) |
| | (SEQ ID NO: 25) | |

IP1 Accumulation Assay. IP1 concentrations in GNAQ-transfected HEK293T cells, GNAQ-expressing melan-a cells and uveal melanoma cells was measured using a competitive homogenous time resolved fluorescence (HTRF) assay (CisBio, Bedford, Mass.). Briefly, 9,000 transiently transfected-HEK293T cells, 5,000 melan-a cells and 5,000 uveal melanoma cells were seeded in low-volume 384-well plates in 7 μL media for 24 hours prior YM-254890 treatment. Cells were then treated with various concentrations of YM-254890 diluted in the appropriate media at 37° C. for 3 hours or 24 hours. 1 h prior lysis, cells were supplemented with 1× Stimulation Buffer provided by the manufacturer (HEPES 10 mM, CaCl₂) 1 mM, MgCl₂ 0.5 mM, KCl 4.2 mM, NaCl 146 mM, glucose 5.5 mM, LiCl 50 mM, pH 7.4) with 0.2% BSA and 50 mM of LiCl (to prevent IP1 degradation). Following incubation, cells were lysed by adding 3 μL/well of d2-labeled IP1 analogue as the fluorescence acceptor and the Terbium cryptate-labeled anti-IP1 mAb as the fluorescence donor, diluted in the kit-supplied lysis buffer. The plates were incubated overnight at RT and time-resolved fluorescence signals were read using the BioTek Synergy NEO plate reader (BioTek Instruments, Winooski, Vt.) at 620 nm and 655 nm. Results were calculated as a 665 nm/620 nm signal ratio, and IP1 concentrations were interpolated from a standard curve prepared using the supplied IP1 calibrator. Results are shown as IP1 (nM). Dose-response curves and bar graphs were analyzed using GraphPad Prism 7.0 software.

Statistical Analysis. All statistical comparisons were done with Graphpad Prism 7.0 software and used a two-tailed Student's t-test for comparison between groups. Data is shown as the mean±SEM (unless otherwise noted) and a P less than 0.05 was used to designate significance.

### Example 2: UVM Patients Harbor Activating Mutations in Gαq Proteins

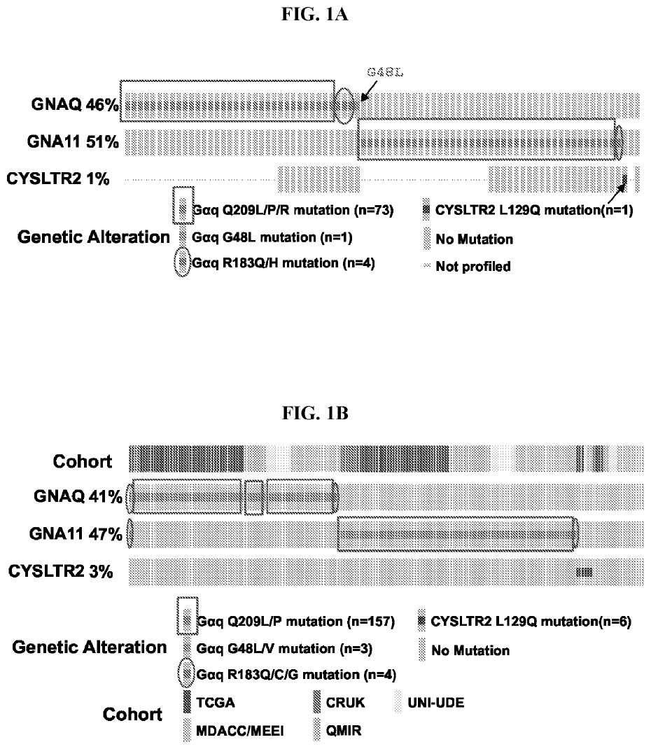

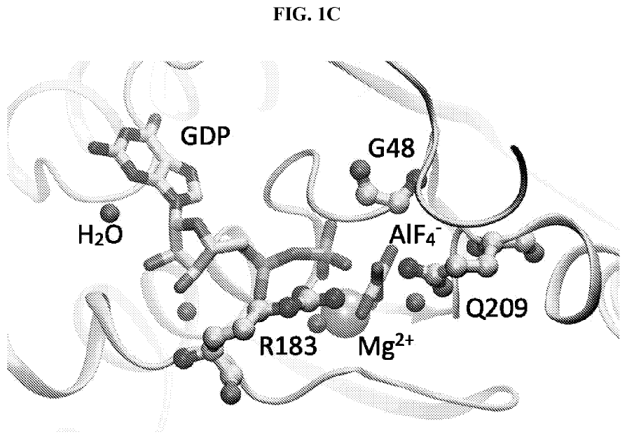

The Gαq pathway mutations from a consecutive series of 81 patients with uveal melanoma that had undergone clinical sequencing on the MSK-IMPACT platform were examined. Similar to previously reported cohorts, 78 of 81 patients harbored mutually-exclusive activating mutations in either GNAQ or GNA11 and one patient had the CYSLTR2^{L129Q} mutation (FIG. 1(A)). For GNAQ, in addition to recurrent mutations at amino acids Q209 and R183, one sample harbored a G48L mutation.

Next, 188 uveal melanoma patients from 5 published whole-exome or whole-genome uveal melanoma cohorts were integrated (Janes M R, et al., Cell 172(3):578-589 (2018); Zehir A et al., Nat Med. 23(6):703-713 (2017); Robertson, A. et al., Cancer Cell 32(2):204-220 (2017); Furney S J et al., Cancer Discov. 3(10):1122-1129 (2013); and Johansson P, et al., Oncotarget 7(4):4624-4631 (2016)). Two samples with GNAQ^{G48L} and one sample with GNAQ^{G48V} mutation were identified, indicating that G48 is a third mutational hotspot in GNAQ (FIG. 1(B)). The G48 residue resides in the phosphate binding loop (P-loop) of Gαq and is paralogous to G12 in RAS GTPases commonly mutated in cancer. Further, the paralogous G47V mutation in GNAS exhibits constitutive activity (Graziano M P & Gilman A G, J Biol Chem. 264(26):15475-15482 (1989)). Structural studies of active GNAQ indicate that G48 of the P-loop, R183 of Switch 1, and Q209 of Switch 2 are in spatial proximity adjacent to the nucleotide-binding pocket (FIG. 1(C)). See also Tesmer V M et al., Science 310(5754):1686-1690 (2005). Thus, Gαq mutations in UVM patients occur at three hotspot residues that are known to affect the guanine-nucleotide binding pocket.

### Example 3: Generation of Syngeneic Mutation Dependent Melanocytes

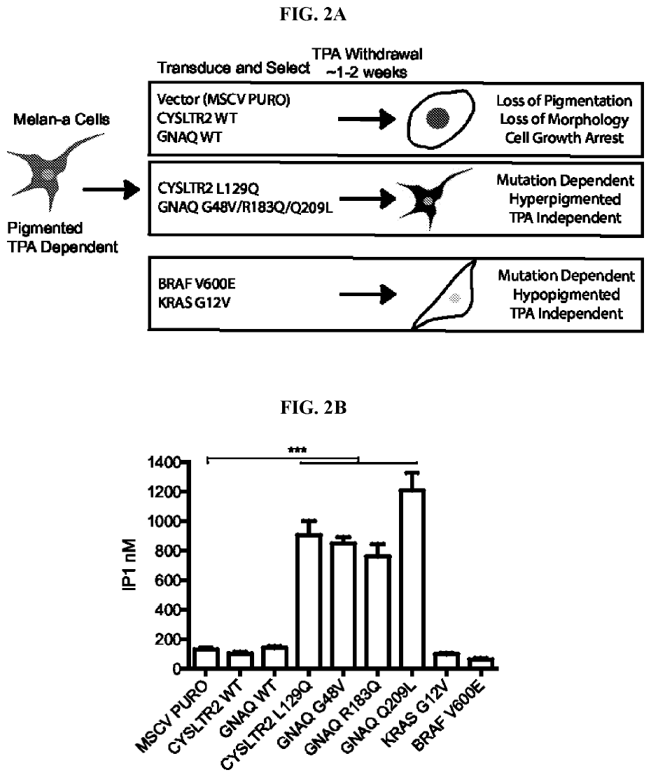

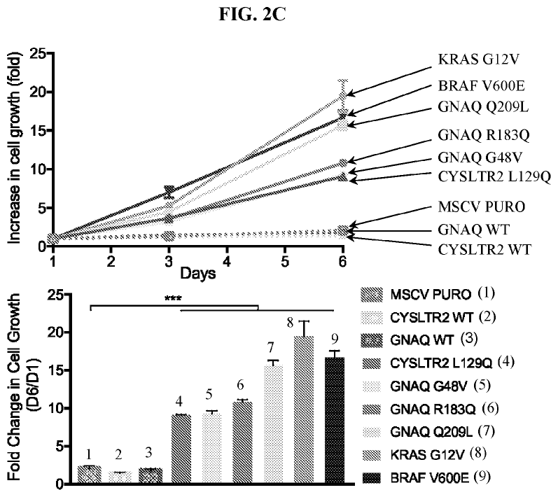

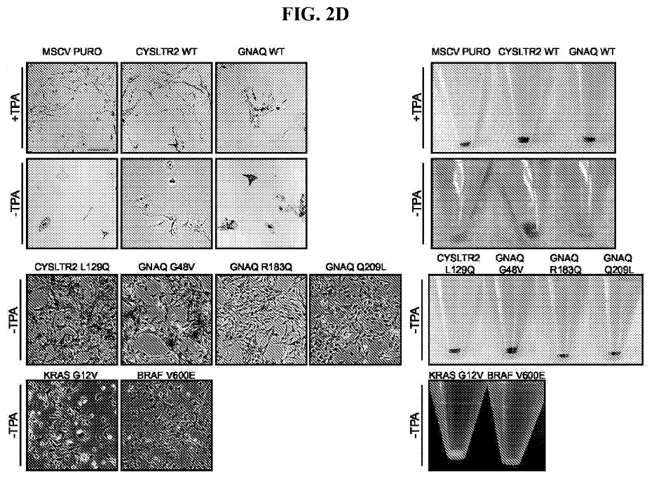

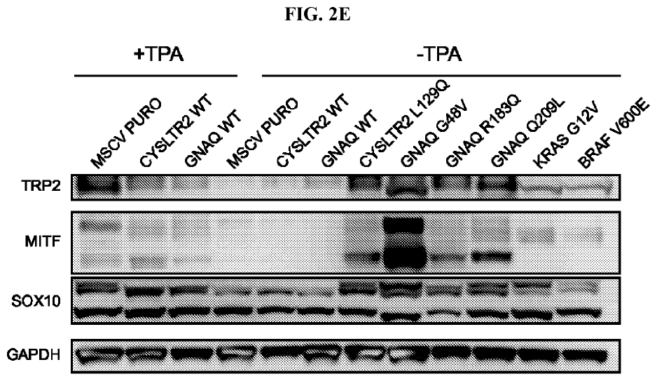

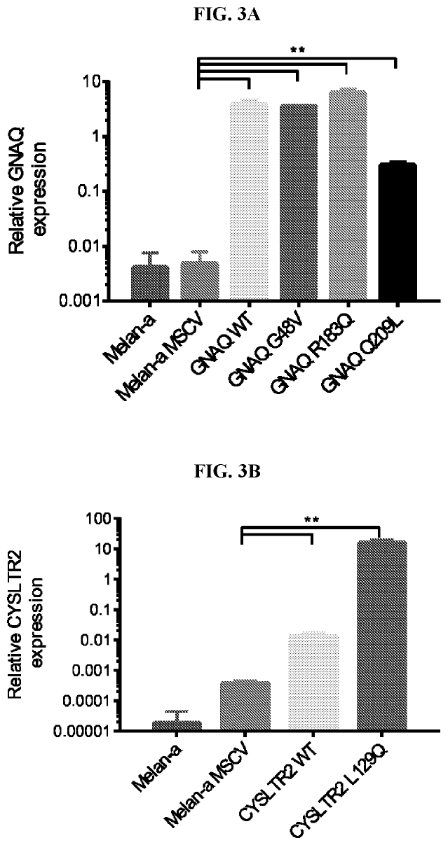

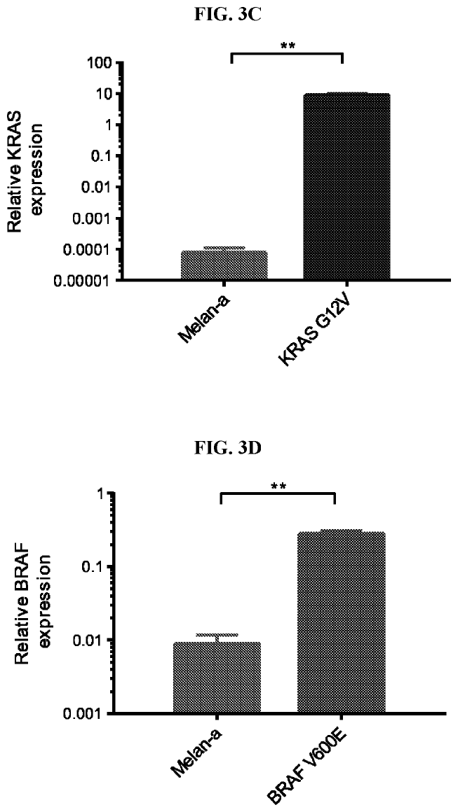

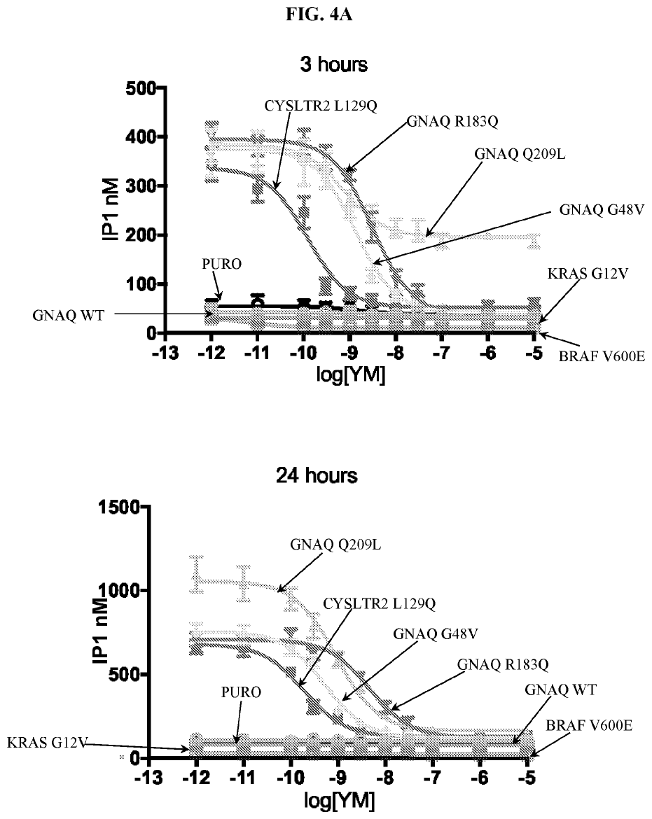

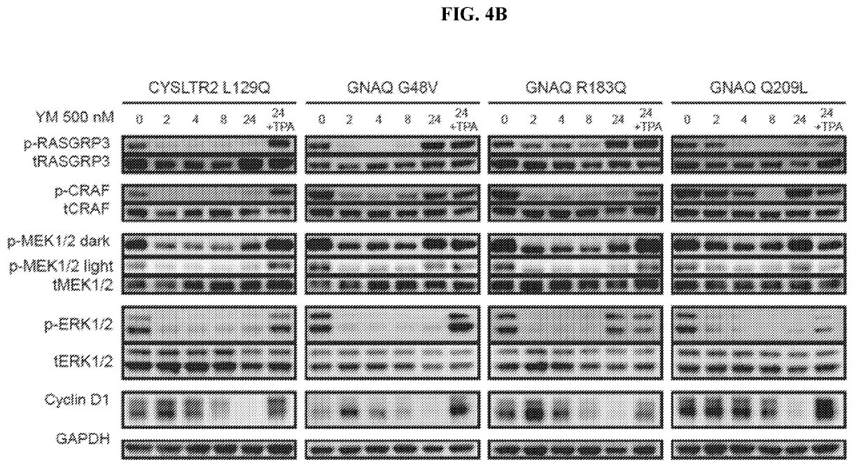

To determine the role of distinct UVM driver oncogenic mutations in a genetically-defined context, human cDNAs encoding CYSLTR2^{L129Q}, GNAQ^{G48V}, GNAQ^{R183Q}, and GNAQ^{Q209L}, WT controls (CYSLTR2^{WT} and GNAQ^{WT}), as well as BRAF^{V600E} and KRAS^{G12V} were stably expressed in melan-a cells (FIG. 2(A), FIG. 3(A)-3(D)). BRAF^{V600E} and KRAS^{G12V} mutants are generally found in cutaneous melanoma and activate the RAS-RAF-MAP kinase signaling pathway, which is distinct from the GPCR-Gαq-PLCβ signaling pathway. Melan-a cells are immortalized mouse melanocytes that require media supplemented with 12-O-tetradecanoyl-phorbol-13-acetate (TPA) for continued proliferation and are characterized by pigmentation and melanocytic morphology. To determine the ability of Gαq to stimulate phospholipase Cβ and generate inositol 1,4,5-triphosphate (IP3), accumulation of the IP3 degradation product IP1 was measured. Melan-a cells expressing CYSLTR2^{L129}G and all three GNAQ mutations exhibited enhanced IP1 accumulation, whereas the WT-, BRAF^{V600E}-, and KRAS^{G12V}-expressing cells did not, demonstrating that this cellular system faithfully recapitulated the distinct signaling pathways driven by CYSLTR2 and Gαq oncoproteins (FIG. 2(B)).

The growth and phenotype of these melan-a cells were examined. After TPA withdrawal, cells expressing vector or WT controls lost pigmentation, rounded up, and eventually arrested in growth. However, cells expressing activating mutations of GNAQ and CYSLTR2 proteins exhibited TPA-independent growth and enhanced melanocytic features, e.g., dark pigmentation. On the other hand, mutations that hyperactivated MAPK signaling (KRAS^{G12V} and BRAF^{V600E}) conveyed TPA-independence, but the cells lost pigmentation (FIG. 2(C)-2(D)). Immunoblot analysis of melan-a cells after TPA-withdrawal showed that UVM associated CYSLTR2 and Gαq oncoproteins maintained expression of melanocyte markers MITF, SOX10, and TRP2/DCT, whereas WT controls, KRAS^{G12V} and BRAF^{V600E} did not (FIG. 2(E)). These data indicate that Gag activation generates a distinct oncogenic phenotype that maintains melanocyte lineage specification, and is consistent with previous observations in genetically engineered murine models of UVM (Moore A R, et al. Cell Rep. 22(9):2455-2468 (2018); Huang J L Y et al., Cancer Res. 75(16):3384-3397 (2015)).

These results demonstrate that the engineered mutant oncoprotein-transformed melanocytes of the present technmology permits systematic evaluation of clinical Gαq-pathway activating mutations. Accordingly the recombinant melanin producing cells of the present technology are useful in methods for identifying candidate agents for treating activated Gαq mutant cancers (e.g., melanoma or angiosarcoma) or activated Gαq mutant melanocytic malignancies (e.g., Portwine stain or Sturge-Weber syndrome).

### Example 4: YM-254890 Inhibits Mutation Dependent Melan-a Cell Growth and Signaling

The sensitivity of distinct mutations to YM treatment was determined. Without wishing to be bound by theory, it is believed that YM stabilizes the GDP-bound state of Gαq^{WT}, and that mutations upstream of WT Gαq may be sensitive to YM treatment.

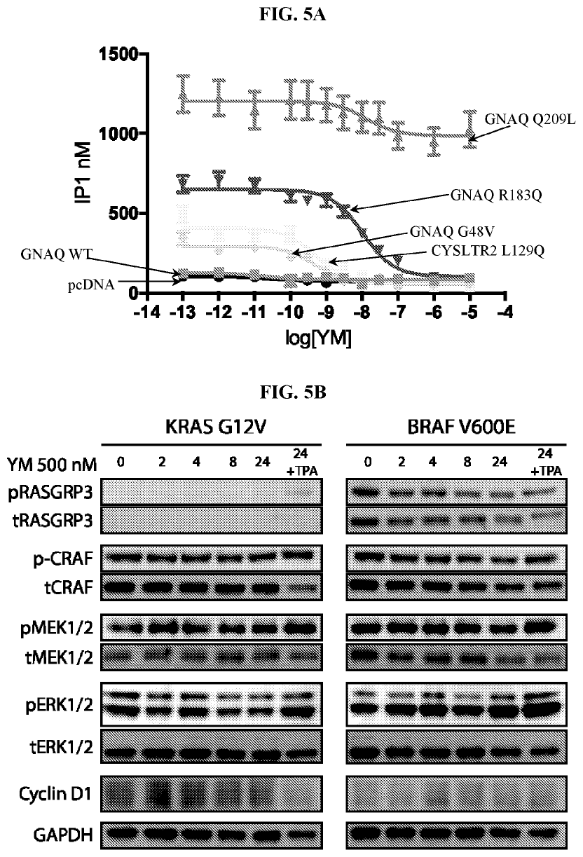

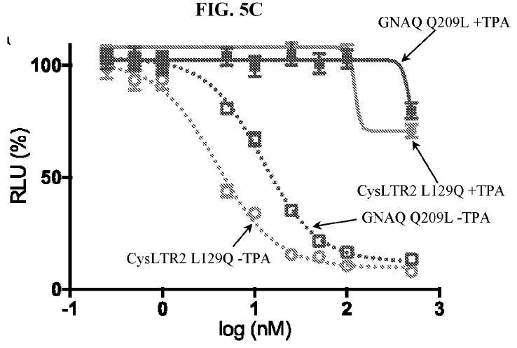

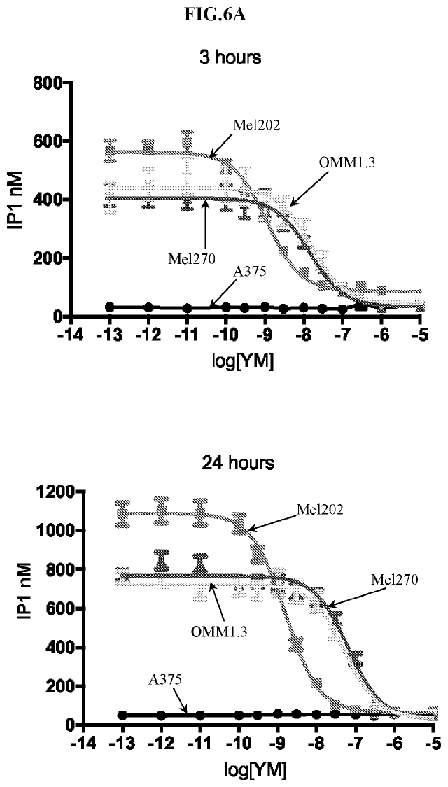

To test the ability of YM to inhibit signaling of Gαq pathway oncoproteins in a conventional system, HEK293T cells were transfected with cDNA constructs and assayed for IP1 accumulation after 24-hour treatment with variable doses of YM. In this system, CYSLTR2^{L129Q} and GNAQ^{G48V} oncoproteins appeared to be most sensitive to YM with subnanomolar potency; GNAQ^{R183Q} was ˜20-fold less sensitive to YM compared to CYSLTR2^{L129Q} and GNAQ^{G48V} GNAQ^{Q209L} was the most resistant to YM and its signaling was incompletely/poorly inhibited within 24-hour YM treatment, which is consistent with previous reports (FIG. 5A) (Takasaki J, et al., J Biol Chem. 279(12):47438-47445 (2004)).

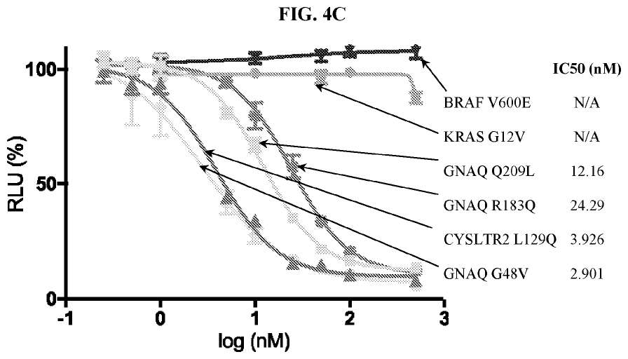

Transient transfection experiments in HEK293T cells results in non-physiologic overexpression of oncoproteins and can result in misleading observations. Accordingly, physiological cellular context is necessary for accurate understanding and characterization of the various hotspot mutations in Gαq signaling pathways in UVM. In the more physiologically relevant context of Gαq pathway mutant-transformed melan-a cells, 24-hour treatment with YM completely inhibited IP1 accumulation across all UVM activating mutants, including the GNAQ^{Q209L} (FIG. 4A). CYSLTR2^{L129Q} and GNAQ^{G48V} mutants were most sensitive to YM, whereas GNAQ^{R183Q} and GNAQ^{Q209L} mutants were approximately 10-fold less sensitive. Interestingly, when IP1 was assayed after 3 hours of treatment, Gαq mutants appeared less sensitive and the GNAQ^{Q209L} mutant were only partially inhibited even at high doses of YM. These data demonstrate that these activating Gαq mutants have the ability to hydrolyze GTP in the relevant cellular system because the YM preferentially access and stabilizes the GDP-bound Gαq state, reminiscent of KRAS^{G12C} inhibition by ARS853. The differential sensitivity of Gag mutants to YM indicate that different Gag mutants have different GTPases activity which can be influenced by the intrinsic GTPases activity of the Gαq mutants, the differential rate of nucleotide exchange mediated by GEF (e.g., GPCR), and other regulatory factors in the relevant cellular context.

The effect of YM on Gαq downstream signaling was assayed, including RASGRP3 phosphorylation by PKC, phosphorylation of CRAF, MEK, and ERK (downstream of RAS) and cyclin D1 expression which integrates signaling to promote cell cycle progression. YM inhibited these downstream signaling targets, with slower kinetics in GNAQ^{R183Q} and GNAQ^{Q209L}-expressing cells compared to CYSLTR2^{L129Q} and GNAQ^{G48V}-expressing cells (FIG. 4B), consistent with the results of the IP1 biochemical assay shown in FIG. 4(A). TPA supplementation completely rescued these downstream signaling targets 24 hours after YM treatment. YM was ineffective in inhibiting MAPK signaling in KRAS^{G12V}- and BRAF^{V600E}-expressing cells (FIG. 5B), indicating that the effect of YM is specific to Gαq inhibition.

The effect of YM on TPA-independent cell growth in the engineered melan-a cells was examined. The CYSLTR2^{L129Q} and GNAQ^{G48V}-expressing cells exhibited sub-nanomolar sensitivity to YM, and the GNAQ^{R183Q} and GNAQ^{Q209L}-expressing cells were modestly less sensitive, whereas the KRAS^{G12V} and BRAF^{V600E}-expressing cells were completely resistant to YM (FIG. 4C). TPA rescued YM-mediated growth inhibition in CYSLTR2^{L129Q} and GNAQ^{Q209L}-expressing cells (FIG. 5C). These data indicate that YM is highly selective in inhibiting tumorigenic signaling and growth mediated by a broad-spectrum of CYSLTR2 and Gαq oncoproteins.

These results demonstrate that YM-254890 is useful in methods for treating activated Gαq mutant cancers (e.g., melanoma or angiosarcoma) or activated Gαq mutant melanocytic malignancies (e.g., Portwine stain or Sturge-Weber syndrome).

### Example 5: Gαq Inhibition Perturbs UVM Cell Growth and Signaling

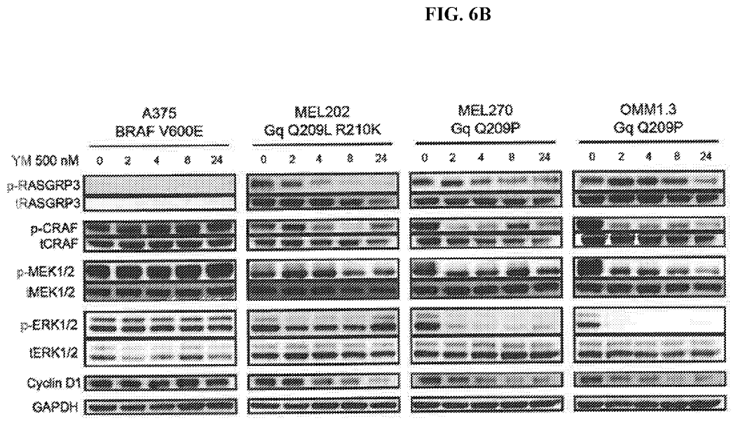

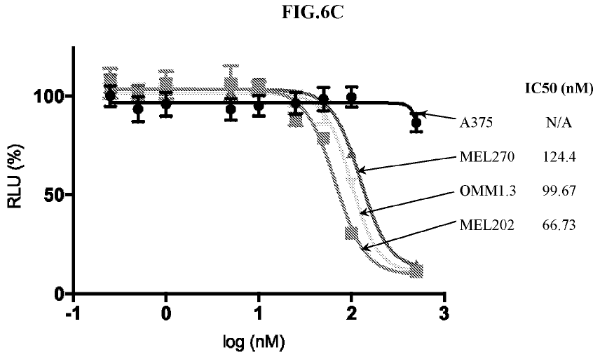

Given the unexpected sensitivity of GNAQ^{Q209L} mutation to YM in the melan-a system, the effect of YM on three human UVM cell lines that harbored the GNAQ^{Q209L/P} mutation was evaluated, using the BRAF^{V600E} mutant A375 cutaneous melanoma cell line as a negative control. UVM cells exhibited high basal activity of IP1 accumulation, which was inhibited upon YM treatment at 24 hours (FIG. 6(A)) Analysis of downstream signaling showed that PKC and MAPK signaling, and cyclin D1 protein were inhibited in UVM cells, but not in A375 cells (FIG. 6(B)). Mel270 and OMM1.3 cells showed small decreases in pRASGRP3 at 24 hours although MAPK targets (p-CRAF, p-MEK1/2, and p-ERK1/2) showed significant inhibition at 2 hours. Mel202 cells, in contrast, showed greater inhibition of pRASGRP3 at earlier time points, but decreased inhibition of MAPK targets with rebound of p-ERK1/2 at 24 hours.

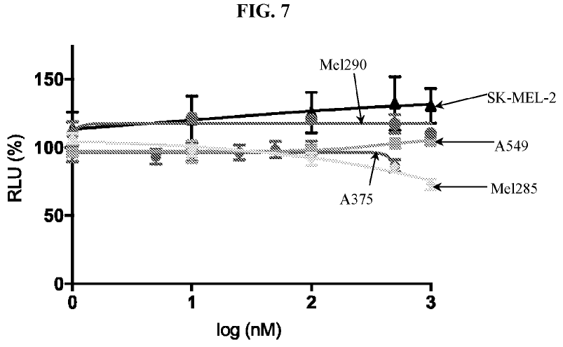

The ability of YM to inhibit cell proliferation was assessed. In all three UVM lines, YM inhibited proliferation with an approximate IC₅₀ of 100 nM, whereas A375 and five other cancer cell lines including two UVM lines that lack GNAQ/11 mutations (Mel285 and Mel290) were resistant to YM (FIG. 6(C), FIG. 7)).

These results demonstrate that the cyclic depsipeptide YM-254890 is useful in methods for treating activated Gαq mutant cancers (e.g., melanoma or angiosarcoma) or activated Gαq mutant melanocytic malignancies (e.g., Portwine stain or Sturge-Weber syndrome).

### Example 6: Gαq Inhibition Perturbs UVM Cell Growth and Signaling

The effect of FR900359 on human UVM cell lines that harbor the GNAQ^{Q209L/P} mutation will be evaluated, using the BRAF^{V600E} mutant A375 cutaneous melanoma cell line as a negative control. It is anticipated that FR900359 treatment will inhibit IP1 accumulation, PKC signaling, MAPK signaling, and/or cyclin D1 protein in UVM cells, but not in A375 cells. The ability of FR900359 to inhibit cell proliferation will be assessed. It is anticipated that FR900359 treatment will inhibit proliferation of human UVM cell lines, but not 375 cells.

These results demonstrate that the cyclic depsipeptide FR900359 is useful in methods for treating activated Gαq mutant cancers (e.g., melanoma or angiosarcoma) or activated Gαq mutant melanocytic malignancies (e.g., Portwine stain or Sturge-Weber syndrome).

### Example 7: Synergistic Effects of Combination Therapy with YM-254890 Synergistic MEKi

To investigate the signaling pathways in which Gq is involved, quantitative phospho-Mass Spec and RNA-seq analysis were performed. It was observed that Gq predominantly signals through the MAP kinase pathway. Therefore, synergy of YM-254890 with FDA approved MEK inhibitors trametinib and binimetinib was tested.

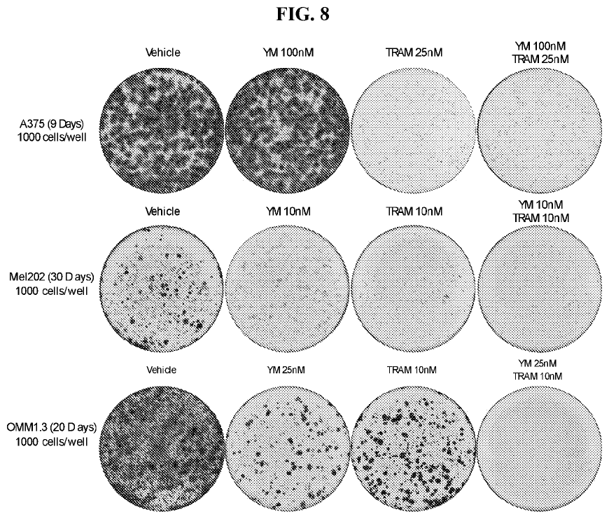

To evaluate the effect of YM-254890 and trametinib in vitro, colony formation assays were performed. 1000 cells per well of the following cell lines were plated: (1) BRAF-mutant A375 cutaneous melanoma cells, (2) Gq-mutant Mel202 cells, and (3) Gq-mutant OMIM1.3 uveal melanoma cells. The cells were then treated with a negative vehicle control, YM-254890, trametinib, or both. Cells were allowed to grow until visible colonies formed in the vehicle-treated plates. As shown in FIG. 8, A375 cells were resistant to YM-254890, but sensitive to trametinib. Mel202 and OMIM1.3 cells showed moderate sensitivity to YM-254890 and trametinib as evidenced by reduced colony formation. The combination of YM-254890 and trametinib showed synergistic inhibitory effects on colony formation by Mel202 and OMIM1.3 cells.

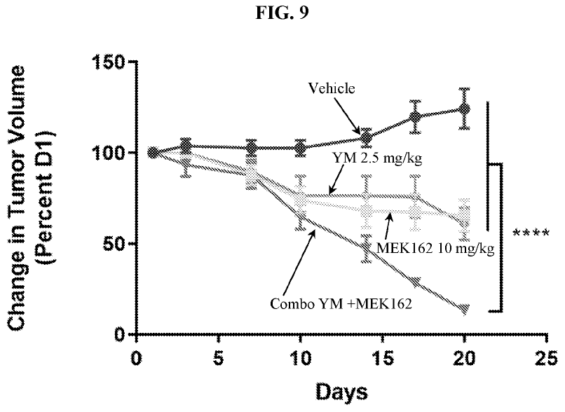

To evaluate the effect of YM-254890 and binimetinib (MEK162) in vivo, Mel202 uveal melanoma xenografts were established in SCID mice, and the mice were treated with vehicle only, 10 mg/kg MEK162 (P.O., b.i.d.), YM-254890 (2.5 mg/kg i.p., q.d.) or a combination of MEK162 and YM-254890. Tumor volumes were measured over 20 days. As shown in FIG. 9, treatment with YM-254890 and MEK162 showed a moderate reduction in tumor volumes compared with the negative vehicle control group. The mice that received the combination of YM-254890 and MEK162 showed a significant reduction in tumor volume compared to mice that received the vehicle control, YM-254890 only or MEK162 only (FIG. 9). Visual observation of mice indicated that YM-254890 was tolerated well, with no detectable side effects.

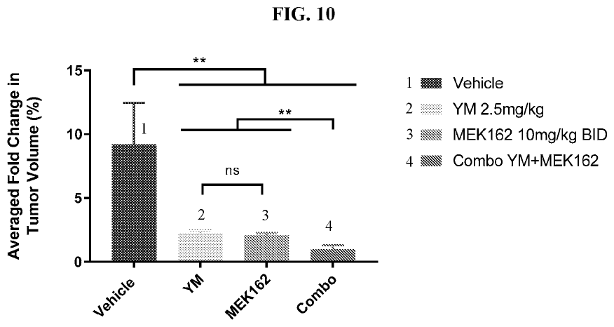

To further evaluate the effect of YM-254890 and MEK162 in vivo, GNAQ^{Q209L} transduced mouse melan-A cells were grafted into C57/B6 mice. Once palpable tumors were established, the mice were treated with vehicle only, 10 mg/kg MEK162 (P.O., b.i.d.), YM-254890 (2.5 mg/kg i.p., q.d.) or a combination of MEK162 and YM-254890. Tumor volumes were measured after 20 days. As shown in FIG. 10, treatment with YM-254890 and MEK162 showed a statistically significant reduction in tumor volume compared with mice that received the vehicle control, YM-254890 only, or MEK162 only. Visual observation of mice indicated that YM-254890 was tolerated well, with no detectable side effects.

These results demonstrate that combination therapy with YM-254890 and MEK162 showed synergistic anti-cancer effects in vitro and in vivo.

These results demonstrate that combination therapy with the cyclic depsipeptides and MEK inhibitors disclosed herein are useful in methods for treating activated Gαq mutant cancers (e.g., melanoma or angiosarcoma) or activated Gαq mutant melanocytic malignancies (e.g., Portwine stain or Sturge-Weber syndrome).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A recombinant melanin producing cell comprising a non-endogenous expression vector comprising a Gαq mutant gene that is operably linked to an expression control sequence, wherein the Gαq mutant gene is selected from the group consisting of GNAQ G48L, GNAQ G48V, GNAQ R183Q, GNAQ R183H, GNAQ R183G, GNA11 G48L, GNA11 G48V, GNA11 R183Q, GNA11 R183H, GNA11 R183G, and CYSLTR2 L129Q.

2. The recombinant melanin producing cell of claim 1, wherein the cell is a melanoblast or a melanocyte.

3. The recombinant melanin producing cell of claim 1, wherein the expression vector is a plasmid, a cosmid, a bacmid, a bacterial artificial chromosome (BAC), a yeast artificial chromosome (YAC), or a viral vector.

4. The recombinant melanin producing cell of claim 1, wherein the expression control sequence is an inducible promoter or a constitutive promoter, or is a native promoter of the Gαq mutant gene or a heterologous promoter.

5. (canceled)

6. A method for identifying a candidate agent for treating an activated GαQ mutant cancer or an activated GαQ mutant melanocytic malignancy or determining an effective amount of a candidate agent for treating an activated GαQ mutant cancer or an activated GαQ mutant melanocytic malignancy comprising
(a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and
(b) detecting expression levels of IP1 and/or cyclin D1 in the recombinant melanin producing cells of step (a),
wherein a reduction in IP1 and/or cyclin D1 expression levels in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy or that the test amount of the candidate agent is effective in treating an activated GαQ mutant cancer or an activated GαQ mutant melanocytic malignancy.

7. A method for identifying a candidate agent for treating an activated GαQ mutant cancer or an activated GαQ mutant melanocytic malignancy or determining an effective amount of a candidate agent for treating an activated GαQ mutant cancer or an activated GαQ mutant melanocytic malignancy comprising
(a) contacting recombinant melanin producing cells with a candidate agent, wherein the recombinant melanin producing cells comprise a first non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q;
(b) contacting recombinant non-melanin producing cells with the candidate agent, wherein the recombinant non-melanin producing cells comprise a second non-endogenous expression vector comprising the same Gαq mutation as the recombinant melanin producing cells of step (a); and
(c) detecting IP1 expression levels in the recombinant melanin producing cells of step (a) and the recombinant non-melanin producing cells of step (b),
wherein a reduction in IP1 expression levels in the recombinant melanin producing cells of step (a) compared to that observed in recombinant non-melanin producing cells of step (b) indicates that the candidate agent is effective in treating an activated GαQ mutant cancer or an activated GαQ mutant melanocytic malignancy or that the test amount of the candidate agent is effective in treating an activated GαQ mutant cancer or an activated GαQ mutant melanocytic malignancy, optionally wherein the method further comprises
detecting phosphorylation of one or more of RASGRP3, CRAF, MEK, and ERK in the recombinant melanin producing cells of step (a) and the recombinant non-melanin producing cells of step (b), or
detecting TPA-independent proliferation in the recombinant melanin producing cells of step (a) and the recombinant non-melanin producing cells of step (b).

8. (canceled)

9. (canceled)

10. A method for identifying a candidate agent for treating an activated GαQ mutant cancer or an activated GαQ mutant melanocytic malignancy or determining an effective amount of a candidate agent for treating an activated GαQ mutant cancer or an activated GαQ mutant melanocytic malignancy comprising
(a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and
(b) detecting phosphorylation of one or more of RASGRP3, CRAF, MEK, and ERK in the recombinant melanin producing cells of step (a),
wherein reduced phosphorylation in one or more of RASGRP3, CRAF, MEK, or ERK in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy or that the test amount of the candidate agent is effective in treating an activated GαQ mutant cancer or an activated GαQ mutant melanocytic malignancy.

11. A method for identifying a candidate agent for treating an activated GαQ mutant cancer or an activated GαQ mutant melanocytic malignancy or determining an effective amount of a candidate agent for treating an activated GαQ mutant cancer or an activated GαQ mutant melanocytic malignancy comprising
(a) contacting recombinant melanin producing cells with a candidate agent, wherein the recombinant melanin producing cells comprise a non-endogenous expression vector comprising a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q; and
(b) detecting TPA-independent proliferation of the recombinant melanin producing cells of step (a),
wherein a reduction in TPA-independent proliferation in the recombinant melanin producing cells of step (a) compared to that observed in recombinant melanin producing cells that are not contacted with the candidate agent indicates that the candidate agent is effective in treating an activated GαQ mutant cancer or an activated GαQ mutant melanocytic malignancy or that the test amount of the candidate agent is effective in treating an activated GαQ mutant cancer or an activated GαQ mutant melanocytic malignancy.

12. (canceled)

13. (canceled)

14. (canceled)

15. (canceled)

16. (canceled)

17. (canceled)

18. The method of claim 6, wherein the activated GαQ mutant cancer or the activated GαQ mutant melanocytic malignancy
is a uveal melanoma, a cutaneous melanoma, a primary meningeal melanocytic tumor, an angiosarcoma, a Portwine stain, or Sturge-Weber syndrome, or
does not harbor a MAPK activating mutation selected from the group consisting of KRAS^{G12V}, KRAS^{G12D}, KRAS^{G12C}, KRAS^{G13D}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

19. (canceled)

20. (canceled)

21. A method for treating an activated GαQ mutant cancer or an activated GαQ mutant melanocytic malignancy in a subject in need thereof comprising administering to the subject a therapeutically effective amount of YM-254890 or FR900359.

22. The method of claim 21 further comprising administering to the subject a therapeutically effective amount of a MEK inhibitor.

23. The method of claim 22, wherein the MEK inhibitor is selected from the group consisting of trametinib, cobimetinib, binimetinib, selumetinib, PD-325901, TAK-733, CI-1040 (PD184352), PD0325901, MEK162, AZD8330, GDC-0623, refametinib, pimasertib, RO4987655, RO5126766, WX-554, HL-085, CInQ-03, G-573, PD184161, PD318088, PD98059, RO5068760, U0126, and SL327.

24. The method of claim 22, wherein the MEK inhibitor and YM-254890 or FR900359 are administered sequentially, simultaneously, or separately.

25. The method of claim 22, wherein the MEK inhibitor and/or the YM-254890 or FR900359 is administered orally, intranasally, intravenously, intramuscularly, intraperitoneally, subcutaneously, intrahepatically, intraarterially, intratumorally, rectally, intracranially, intrathecally, or topically.

26. (canceled)

27. The method of claim 21, wherein the activated GαQ mutant cancer or the activated GαQ mutant melanocytic malignancy is a uveal melanoma, a cutaneous melanoma, a primary meningeal melanocytic tumor, an angiosarcoma, a Portwine stain, or Sturge-Weber syndrome; or
comprises a Gαq mutation selected from the group consisting of GNAQ G48L, GNAQ G48V, GNAQ R183Q, GNAQ R183H, GNAQ R183G, GNAQ R183C, GNAQ Q209L, GNAQ Q209P, GNAQ Q209R, GNA11 G48L, GNA11 G48V, GNA11 R183Q, GNA11 R183H, GNA11 R183G, GNA11 R183C, GNA11 Q209L, GNA11 Q209P, GNA11 Q209R, and CYSLTR2 L129Q, or
does not harbor a MAPK activating mutation selected from the group consisting of KRAS^{G12V}, KRAS^{G12D}, KRAS^{G12C}, KRAS^{Gt3D}, KRAS^{Q61H}, HRAS^{G12V}, HRAS^{G13R}, HRAS^{Q61R}, NRAS^{G12D}, NRAS^{G13D}, NRAS^{Q61A}, NRAS^{Q61L}, NRAS^{Q61L}, BRAF^{V600K} or BRAF^{V600E}.

28. (canceled)

29. (canceled)

30. (canceled)

31. The method of claim 21, further comprising sequentially, separately, or simultaneously administering one or more therapeutic agents selected from the group consisting of: alkylating agents, antimetabolites, natural products, hormones, platinum coordination complexes, anthracenedione, substituted urea, methyl hydrazine derivatives, and adrenocortical suppressants.

32. A kit comprising
cyclic depsipeptide YM-254890 or FR900359, and instructions for treating an activated GαQ mutant cancer or an activated GαQ mutant melanocytic malignancy, or
the recombinant melanin producing cell of claim 1 and instructions for assaying the effectiveness of a candidate agent for treatment of an activated GαQ mutant cancer or an activated GαQ mutant melanocytic malignancy.

33. (canceled)

34. The kit of claim 32, wherein the activated GαQ mutant cancer or the activated GαQ mutant melanocytic malignancy is a uveal melanoma, a cutaneous melanoma, a primary meningeal melanocytic tumor, an angiosarcoma, a Portwine stain, or Sturge-Weber syndrome.
